(19) United States
(12) Patent Application Publication
(10) Pub. No.: US 2025/0292866 A1
(43) Pub. Date: Sep. 18, 2025

(54) METHOD AND APPARATUS FOR PREDICTING AFFINITY OF ANTIBODY SEQUENCE FOR ANTIGEN SEQUENCE, COMPUTER DEVICE, AND STORAGE MEDIUM

(71) Applicant: Tencent Technology (Shenzhen) Company Limited, Shenzhen, (CN)

(72) Inventors: Bing HE, Shenzhen (CN); Haohuai HE, Shenzhen (CN); Jianhua YAO, Shenzhen (CN); Yu ZHAO, Shenzhen (CN)

(73) Assignee: Name Tencent Technology (Shenzhen) Company Limited, Shenzhen (CN)

(21) Appl. No.: 19/226,298

(22) Filed: Jun. 3, 2025

Related U.S. Application Data
(63) Continuation of application No. PCT/CN2024/082139, filed on Mar. 18, 2024.

(30) Foreign Application Priority Data
May 18, 2023 (CN) 202310562554.X

(51) Int. Cl. G16B 15/30; G06F 30/27; G16B 40/20

(52) U.S. Cl. CPC G16B 15/30; G06F 30/27; G16B 40/20

(57) ABSTRACT

A method, apparatus, and computer-readable storage medium for predicting the affinity of an antibody sequence for an antigen sequence. The method includes obtaining antigen sequence information and antibody sequence information comprising light chain and heavy chain sequence information. Sequence features are extracted from the antigen sequence, light chain sequence, and heavy chain sequence. These extracted features are fused to obtain fused sequence features, which are then processed using a fully connected neural network. The processing determines an affinity detection result representing the affinity of the antibody sequence for the antigen sequence. This computational approach enables efficient prediction of antibody-antigen binding affinities for applications in therapeutic antibody development and immunological research.

### CROSS-REFERENCE TO RELATED APPLICATIONS

This application is a continuation application of International Application No. PCT/CN2024/082139 filed on Mar. 18, 2024 which claims priority to Chinese Patent Application No. 202310562554.X, filed with the China National Intellectual Property Administration on May 18, 2023, the disclosures of each being incorporated by reference herein in their entireties.

### FIELD

The disclosure relates to the field of computer technologies, a method, and apparatus for predicting an affinity of an antibody sequence for an antigen sequence, a computer device, and a storage medium.

### BACKGROUND

An antigen sequence is a substance to be removed by an immune system. The antigen sequence can induce a body to generate an immune response, and be bound to an antibody sequence generated in the immune response, so as to generate an immune defense, thereby playing an important role in maintaining human health. Researching a size of an affinity of the antibody sequence for an antigen sequence is of great importance for understanding the immune system, and may further facilitate immunotherapy and the design and development of a vaccine. Based on this, a method for predicting an affinity of an antibody sequence for an antigen sequence is urgently needed.

### SUMMARY

Provided are a method and apparatus for predicting an affinity of an antibody sequence for an antigen sequence, a device, a storage medium, and a program product, which can implement prediction of antibody-antigen binding affinity using neural network processing of sequence features.

According to some embodiments, a method for predicting an affinity of an antibody sequence for an antigen sequence, performed by a computer device, includes: obtaining antigen sequence information representing an amino acid in an antigen sequence; obtaining antibody sequence information comprising light chain sequence information and heavy chain sequence information of an antibody sequence, wherein the light chain sequence information represents an amino acid in a light chain of the antibody sequence, and the heavy chain sequence information represents an amino acid in a heavy chain of the antibody sequence; extracting an antigen sequence feature from the antigen sequence information; extracting a light chain sequence feature from the light chain sequence information; extracting a heavy chain sequence feature from the heavy chain sequence information; fusing the antigen sequence feature, the light chain sequence feature, and the heavy chain sequence feature, to obtain fused sequence features; processing the fused sequence features based on a fully connected neural network; and determining an affinity detection result representing an affinity of the antibody sequence for the antigen sequence.

According to some embodiments, an apparatus for predicting an affinity of an antibody sequence for an antigen sequence, includes: at least one memory configured to store program code; and at least one processor configured to read the program code and operate as instructed by the program code, the program code including: obtaining code configured to cause at least one of the at least one processor to obtain antigen sequence information representing an amino acid in an antigen sequence; obtaining code configured to cause at least one of the at least one processor to obtain antibody sequence information comprising light chain sequence information and heavy chain sequence information of an antibody sequence, wherein the light chain sequence information represents an amino acid in a light chain of the antibody sequence, and the heavy chain sequence information represents an amino acid in a heavy chain of the antibody sequence; extracting code configured to cause at least one of the at least one processor to extract an antigen sequence feature from the antigen sequence information; extracting code configured to cause at least one of the at least one processor to extract a light chain sequence feature from the light chain sequence information; extracting code configured to cause at least one of the at least one processor to extract a heavy chain sequence feature from the heavy chain sequence information; fusing code configured to cause at least one of the at least one processor to fuse the antigen sequence feature, the light chain sequence feature, and the heavy chain sequence feature, to obtain fused sequence features; processing code configured to cause at least one of the at least one processor to process the fused sequence features based on a fully connected neural network; and determining code configured to cause at least one of the at least one processor to determine an affinity detection result representing an affinity of the antibody sequence for the antigen sequence.

According to some embodiments, a non-transitory computer-readable storage medium, storing computer code which, when executed by at least one processor, causes the at least one processor to at least: obtain antigen sequence information representing an amino acid in an antigen sequence; obtain antibody sequence information comprising light chain sequence information and heavy chain sequence information of an antibody sequence, wherein the light chain sequence information represents an amino acid in a light chain of the antibody sequence, and the heavy chain sequence information represents an amino acid in a heavy chain of the antibody sequence; extract an antigen sequence feature from the antigen sequence information; extract a light chain sequence feature from the light chain sequence information; extract a heavy chain sequence feature from the heavy chain sequence information; fuse the antigen sequence feature, the light chain sequence feature, and the heavy chain sequence feature, to obtain fused sequence features; process the fused sequence features based on a fully connected neural network; and determine an affinity detection result representing an affinity of the antibody sequence for the antigen sequence.

### BRIEF DESCRIPTION OF THE DRAWINGS

To describe the technical solutions of some embodiments of this disclosure more clearly, the following briefly introduces the accompanying drawings for describing some embodiments. The accompanying drawings in the following description show only some embodiments of the disclosure, and a person of ordinary skill in the art may still derive other drawings from these accompanying drawings without creative efforts. In addition, one of ordinary skill would understand that aspects of some embodiments may be combined together or implemented alone.

FIG. 1 is a schematic diagram of an implementation environment according to some embodiments.

FIG. 2 is a schematic diagram of combination of an antigen sequence and an antibody sequence according to some embodiments.

FIG. 3 is a flowchart of a method for predicting an affinity of an antibody sequence for an antigen sequence according to some embodiments.

FIG. 4 is a schematic diagram of a structure of an affinity prediction model according to some embodiments.

FIG. 5 is a flowchart of a method for predicting an affinity of an antibody sequence for an antigen sequence according to some embodiments.

FIG. 6 is a schematic diagram of extraction of an antigen sequence feature according to some embodiments.

FIG. 7 is a schematic diagram of a structure of a multimodal fusion convolutional neural network (MF-CNN) model according to some embodiments.

FIG. 8 is a flowchart of a training method of an affinity prediction model according to some embodiments.

FIG. 9 is a flowchart of a pretraining method of an antigen encoding network according to some embodiments.

FIG. 10 is a flowchart of a pretraining method of a light chain encoding network according to some embodiments.

FIG. 11 is a flowchart of a pretraining method of a light chain encoding network according to some embodiments.

FIG. 12 is a schematic diagram of a performance detection result according to some embodiments.

FIG. 13 is a schematic diagram of another performance detection result according to some embodiments.

FIG. 14 is a schematic diagram of a structure of an apparatus for predicting an affinity of an antibody sequence for an antigen sequence according to some embodiments.

FIG. 15 is a schematic diagram of a structure of another apparatus for predicting an affinity of an antibody sequence for an antigen sequence according to some embodiments.

FIG. 16 is a schematic diagram of a structure of a terminal according to some embodiments.

FIG. 17 is a schematic diagram of a structure of a server according to some embodiments.

### DESCRIPTION OF EMBODIMENTS

To make the objectives, technical solutions, and advantages of the present disclosure clearer, the following further describes the present disclosure in detail with reference to the accompanying drawings. The described embodiments are not to be construed as a limitation to the present disclosure. All other embodiments obtained by a person of ordinary skill in the art without creative efforts shall fall within the protection scope of the present disclosure.

In the following descriptions, related “some embodiments” describe a subset of all possible embodiments. However, it may be understood that the “some embodiments” may be the same subset or different subsets of all the possible embodiments, and may be combined with each other without conflict. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the phrase “at least one of A, B, and C” includes within its scope “only A”, “only B”, “only C”, “A and B”, “B and C”, “A and C” and “all of A, B, and C.”

The terms “first”, “second”, and the like used in this application may be used for describing various concepts in this specification. However, the concepts are not limited by the terms unless otherwise specified. The terms are merely used for distinguishing one concept from another concept. For example, without departing from the scope of this application, a first semantic feature may be referred to as a second semantic feature, and similarly, the second semantic feature may be referred to as the first semantic feature.

“At least one” means one or more. For example, at least one antibody sequence may be an integer quantity of antibody sequences equal to or greater than one, such as one antibody sequence, two antibody sequences, or three antibody sequences. “A plurality of” means two or more. For example, a plurality of antibody sequences may be an integer quantity of antibody sequences equal to or greater than two, such as two antibody sequences or three antibody sequences. “Each” means each of at least one. For example, each antibody sequence refers to each of a plurality of antibody sequences. If the plurality of antibody sequences are three antibody sequences, each antibody sequence refers to each of the three antibody sequences.

In some embodiments, relevant data such as antigen sequence information and antibody sequence information are involved. In a case that the foregoing embodiments of this application are applied to a product or technology with a permission or consent of a user, and collection, use, and processing of the relevant data need to comply with relevant laws, regulations, and standards of relevant countries and regions.

Artificial intelligence (AI) is a theory, method, technology, and application system that uses a digital computer or a machine controlled by the digital computer to simulate, extend, and expand human intelligence, perceive an environment, obtain knowledge, and use knowledge to obtain an optimal result. In other words, AI is a comprehensive technology in computer science and attempts to understand the essence of intelligence and produce a new intelligent machine that can react in a manner similar to human intelligence. AI is to study the design principles and implementation methods of various intelligent machines, to enable the machines to have the functions of perception, reasoning, and decision-making.

The AI technology is a comprehensive discipline, and relates to a wide range of fields including both hardware-level technologies and software-level technologies. The AI technologies generally include technologies such as a sensor, a dedicated AI chip, cloud computing, distributed storage, a big data processing technology, an operating/interaction system, and electromechanical integration. AI software technologies include a computer vision technology, a speech processing technology, a nature language processing (NLP) technology, machine learning (ML)/deep learning, autonomous driving, intelligent transportation, and other major directions.

NLP is an important direction in the field of computer science and the field of AI. NLP studies various theories and methods that can implement effective communication between people and computers by using natural languages. NLP is a comprehensive science of linguistics, computer science, and mathematics. Therefore, the study in this field relates to natural languages, namely, languages daily used by people, and therefore, the natural languages are closely related to linguistic studies.

ML is a multi-field interdiscipline, and relates to a plurality of disciplines such as a probability theory, statistics, an approximation theory, convex analysis, and an algorithm complexity theory. ML specializes in studying how a computer simulates or implements a human learning behavior to obtain new knowledge or skills, and reorganize an existing knowledge structure, so as to keep improving its performance. ML is the core of AI, is a way to make the computer intelligent, and is applied to various fields of AI. The ML and the deep learning generally include technologies such as an artificial neural network, a confidence network, reinforcement learning, transfer learning, inductive learning, and learning from demonstration.

The following describes, based on an AI technology and an ML technology, a method for predicting an affinity of an antibody sequence for an antigen sequence according to some embodiments.

The method for predicting an affinity of an antibody sequence for an antigen sequence according to some embodiments can be applied to a computer device. In some embodiments, the computer device may be a terminal or a server. In some embodiments, the server is an independent physical server, a server cluster composed of a plurality of physical servers or a distributed system, or a cloud server that provides cloud computing services such as a cloud service, a cloud database, cloud computing, a cloud function, cloud storage, a network service, cloud communication, a middleware service, a domain name service, a security service, a content delivery network (CDN), a big data platform, and an AI platform. In some embodiments, the terminal may be a smartphone, a tablet computer, a notebook computer, a desktop computer, a smart speaker, a smart watch, and the like, but is not limited thereto.

In some embodiments, the computer program involved in some embodiments may be deployed in one computer device for execution, or deployed in a plurality of computer devices at one location for execution, or distributed in a plurality of computer devices at a plurality of locations and connected via a communication network. The plurality of computer devices at the plurality of locations and connected via the communication network may form a blockchain system.

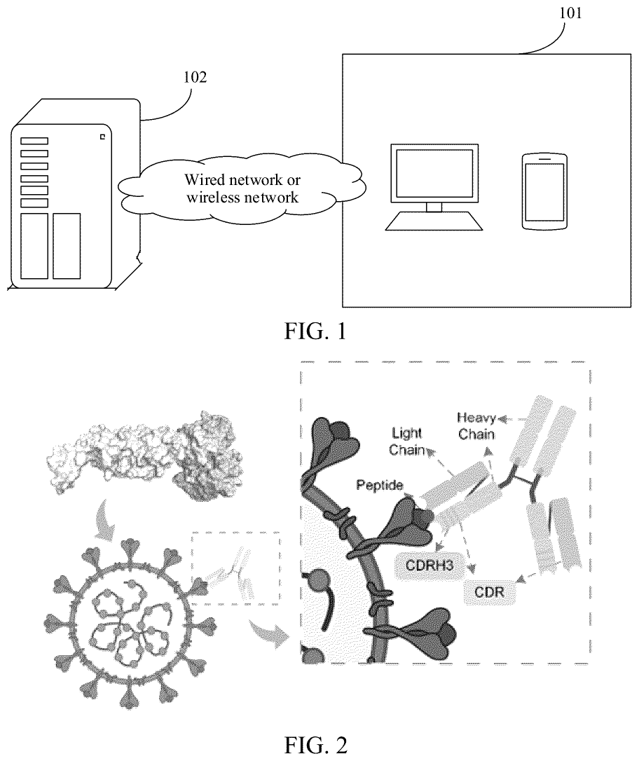

FIG. 1 is a schematic diagram of an implementation environment according to some embodiments. As shown in FIG. 1, the implementation environment includes a terminal 101 and a server 102. The terminal 101 and the server 102 are connected to each other by a wireless or wired network. In some embodiments, the server 102 is configured to train an affinity prediction model. The affinity prediction model is configured to predict an affinity of an antibody sequence for an antigen sequence. The server 102 transmits the trained affinity prediction model to the terminal 101. The terminal 101 may predict, by using the affinity prediction model, the affinity of the antibody sequence for the antigen sequence.

In FIG. 1, only an example in which the server 102 trains the affinity prediction model and transmits the affinity prediction model to the terminal 101 is used for description. In another embodiment, the server may predict, by using the affinity prediction model, the affinity of the antibody sequence for the antigen sequence, and then transmit an affinity detection result obtained through prediction to the terminal 101.

The method for predicting an affinity of an antibody sequence for an antigen sequence according to some embodiments may be applied to any scenario in which the affinity of the antibody sequence for the antigen sequence may be detected.

In the research and development of drugs, it is very important to learn about the affinity of the antibody sequence for the antigen sequence to develop a high-affinity antibody sequence or select a suitable antigen sequence. In immune diagnosis, predicting the affinity of the antibody sequence for the antigen sequence may assist in the research and development of a highly sensitive and diagnosis kit. The high-affinity antibody sequence can be more easily bound to and detect the antigen sequence, thereby improving diagnosis accuracy and sensitivity. Meanwhile, in the research and development of a vaccine, predicting the affinity of the antibody sequence for the antigen sequence may assist in evaluation of a potential protective effect of a vaccine candidate. The high-affinity antibody sequence can be effectively bound to and remove pathogens, thereby improving the protective effect of the vaccine.

FIG. 2 is a schematic diagram of combination of an antigen sequence and an antibody sequence according to some embodiments. As shown in FIG. 2, the antigen sequence includes a heavy chain and a light chain. The light chain and the heavy chain respectively include a series of variable regions and constant regions. The variable region includes a complementary determining region (CDR) of a antigen sequence recognized by an antibody sequence, and includes an important CDR3. The CDR3 is the most diversified and important region in an antibody structure, and plays a key role in antigen recognition by an antibody. CDRH3 is a heavy chain CDR3. An amino group of one amino acid may be condensed with a carboxyl group of another amino acid to form a Peptide. The binding tightness may be measured by using a size of the affinity for the binding between the antigen sequence and the antibody sequence. Assuming that the light chains and the heavy chains of the antigen sequence and the antibody sequence include key information required for describing the binding affinity, the binding affinity of the antigen sequence and the antibody sequence may be predicted by using the light chains and the heavy chains of the antigen sequence and the antibody sequence subsequently.

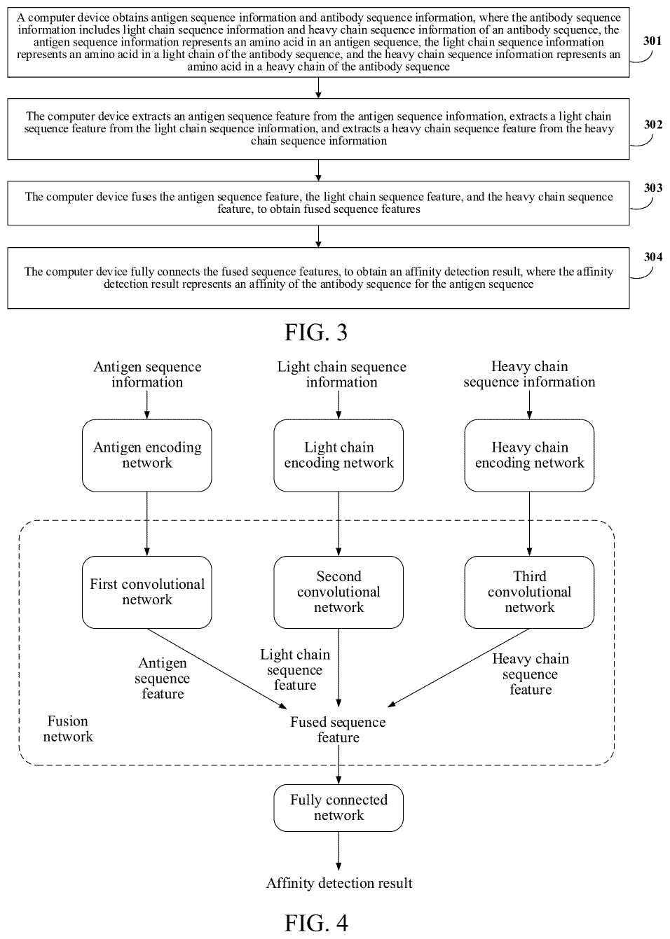

FIG. 3 is a flowchart of a method for predicting an affinity of an antibody sequence for an antigen sequence according to some embodiments. Some embodiments is performed by a computer device. Referring to FIG. 3, the method includes the following operations.

301: The computer device obtains antigen sequence information and antibody sequence information, where the antibody sequence information includes light chain sequence information and heavy chain sequence information of an antibody sequence, the antigen sequence information represents an amino acid in an antigen sequence, the light chain sequence information represents an amino acid in a light chain of the antibody sequence, and the heavy chain sequence information represents an amino acid in a heavy chain of the antibody sequence.

The antigen sequence is a substance to be removed by an immune system. The antigen sequence can induce a body to generate an immune response, and be bound to an antibody sequence generated in the immune response, so as to generate an immune defense (response). The antigen sequence is composed of amino acids. The antibody sequence is a protein synthesized by the immune system, and is also referred to as an immune globulin. The antibody sequence can recognize and be bound to a pathogen invading a human body, such as a bacterium, a virus, or a fungus, and plays an important role in the immune response. The antibody sequence includes a heavy chain and a light chain, and variable regions of the heavy chain and the light chain can be bound to the antigen sequence. The heavy chain is a large polypeptide subunit of the antibody sequence, and the light chain is a small polypeptide subunit of the antibody sequence. Generally, an antibody is composed of four polypeptide chains, namely, two identical light chains having small relative molecular mass and two identical heavy chains having large relative molecular mass. The antibody is a symmetrical structure formed by means of disulfide linkage.

In some embodiments, the computer device obtains antigen sequence information of an antigen sequence and antibody sequence information of an antibody sequence. The antigen sequence and the antibody sequence are an antigen sequence and an antibody sequence between which an affinity is to be predicted. The antigen sequence may be a complete antigen or a part of a sequence in the complete antigen. For example, the antigen sequence may be a part of a sequence composed of amino acids of an epitope of the antigen. The epitope is also referred to as an antigen determinant, and is a position on the antigen that can be bound to an antibody binding site and determine antigen specificity. The antibody sequence may be a complete antibody or a part of a sequence in the complete antibody. For example, the antibody sequence may be a part of a sequence composed of amino acids in a variable region of the antibody. The variable region of the antibody includes a determining region that recognizes a antigen in the antibody.

The antibody sequence information includes light chain sequence information and heavy chain sequence information of the antibody sequence. The light chain sequence information represents an amino acid in a light chain of the antibody sequence. The heavy chain sequence information represents an amino acid in a heavy chain of the antibody sequence. Considering that the light chain and the heavy chain of the antibody sequence respectively include key information required for describing a binding affinity, the sequence information of the light chain and the sequence information of the heavy chain are separately processed in this application.

302: The computer device extracts an antigen sequence feature from the antigen sequence information, extracts a light chain sequence feature from the light chain sequence information, and extracts a heavy chain sequence feature from the heavy chain sequence information.

The computer device performs, after obtaining the antigen sequence information, the light chain sequence information, and the heavy chain sequence information, feature extraction on the antigen sequence information to obtain an antigen sequence feature. The antigen sequence feature indicates types of amino acids in the antigen sequence and a connection structure of the plurality of amino acids. The computer device extracts a light chain sequence feature from the light chain sequence information. The light chain sequence feature represents types of amino acids in the light chain of the antibody sequence and a connection structure of the plurality of amino acids. The computer device extracts a heavy chain sequence feature from the heavy chain sequence information. The heavy chain sequence feature represents types of amino acids in the heavy chain of the antibody sequence and a connection structure of the plurality of amino acids.

The antigen sequence feature, the light chain sequence feature, and the heavy chain sequence feature may be features in a vector form, features in a matrix form, or the like. This is not limited in some embodiments.

303: The computer device fuses the antigen sequence feature, the light chain sequence feature, and the heavy chain sequence feature, to obtain fused sequence features.

After obtaining the antigen sequence feature, the light chain sequence feature, and the heavy chain sequence feature, the computer device fuses the antigen sequence feature, the light chain sequence feature, and the heavy chain sequence feature, to obtain fused sequence features. The fused sequence features include a feature of the antigen sequence, a feature of the light chain, and a feature of the heavy chain, thereby enriching feature expression of the fused sequence features.

304: The computer device fully connects the fused sequence features, to obtain an affinity detection result, where the affinity detection result represents an affinity of the antibody sequence for the antigen sequence.

Because the fused sequence features obtained by the computer device include the feature of the antigen sequence, the feature of the light chain, and the feature of the heavy chain, and the antigen sequence, the light chain, and the heavy chain include the key information required for describing the binding affinity, the fused sequence features are fully connected to obtain an affinity detection result of the antigen sequence and the antibody sequence. The process of fully connecting the fused sequence features is a process of performing affinity prediction based on the fused sequence features, or a process of performing regression based on the fused sequence features, to obtain the affinity detection result of the antigen sequence and the antibody sequence.

In the method according to some embodiments, when an affinity of an antibody sequence for an antigen sequence is predicted, a feature of an amino acid in the antigen sequence, a feature of an amino acid in a light chain of the antibody sequence, and a feature of an amino acid in a heavy chain of the antibody sequence are comprehensively considered. In terms of the heavy chain and the light chain, potential associations of the feature of the amino acid in the antigen sequence with the feature of the amino acid in the light chain and the feature of the amino acid in the heavy chain are considered, so as to predict the affinity of the antibody sequence for the antigen sequence. Not only factors are considered comprehensively, but also two granularities of the heavy chain and the light chain are divided, which is beneficial to improving the accuracy of affinity prediction.

In another embodiment, an affinity prediction model is stored in the computer device. The affinity prediction model is configured to predict an affinity of an antibody sequence for an antigen sequence. FIG. 4 is a schematic diagram of a structure of an affinity prediction model according to some embodiments. As shown in FIG. 4, the affinity prediction model includes: an antigen encoding network, a light chain encoding network, a heavy chain encoding network, a fusion network, and a fully connected network.

The antigen encoding network, the light chain encoding network, and the heavy chain encoding network are respectively connected to the fusion network, and the fusion network is connected to the fully connected network. The antigen encoding network is configured to extract a feature of the antigen sequence information. The light chain encoding network is configured to extract a feature of the light chain sequence information. The heavy chain encoding network is configured to extract a feature of the heavy chain sequence information. The fusion network is configured to fuse features extracted from the antigen encoding network, the light chain encoding network, and the heavy chain encoding network. The fully connected network is configured to fully connect the features, to predict the affinity detection result.

In some embodiments, the fusion network includes a first convolutional network, a second convolutional network, and a third convolutional network. The first convolutional network is connected to the antigen encoding network, and the first convolutional network is configured to convolve the antigen sequence feature outputted by the antigen encoding network, to obtain a deep antigen sequence feature. The second convolutional network is connected to the light chain encoding network, and the second convolutional network is configured to convolve the light chain sequence feature outputted by the light chain encoding network, to obtain a deep light chain sequence feature. The third convolutional network is connected to the heavy chain encoding network, and the third convolutional network is configured to convolve the heavy chain sequence feature outputted by the heavy chain encoding network, to obtain a deep heavy chain sequence feature.

As shown in FIG. 4, an input of the antigen encoding network is the antigen sequence information. An input of the light chain encoding network is the light chain sequence information. An input of the heavy chain encoding network is the heavy chain sequence information. The first convolutional network, the second convolutional network, and the third convolutional network respectively output the deep antigen sequence feature, the deep light chain sequence feature, and the deep heavy chain sequence feature obtained through convolution, and then fuse the deep antigen sequence feature, the deep light chain sequence feature, and the deep heavy chain sequence feature, to obtain fused sequence features. An input of the fully connected network is the fused sequence features. An output of the fully connected network is the affinity detection result.

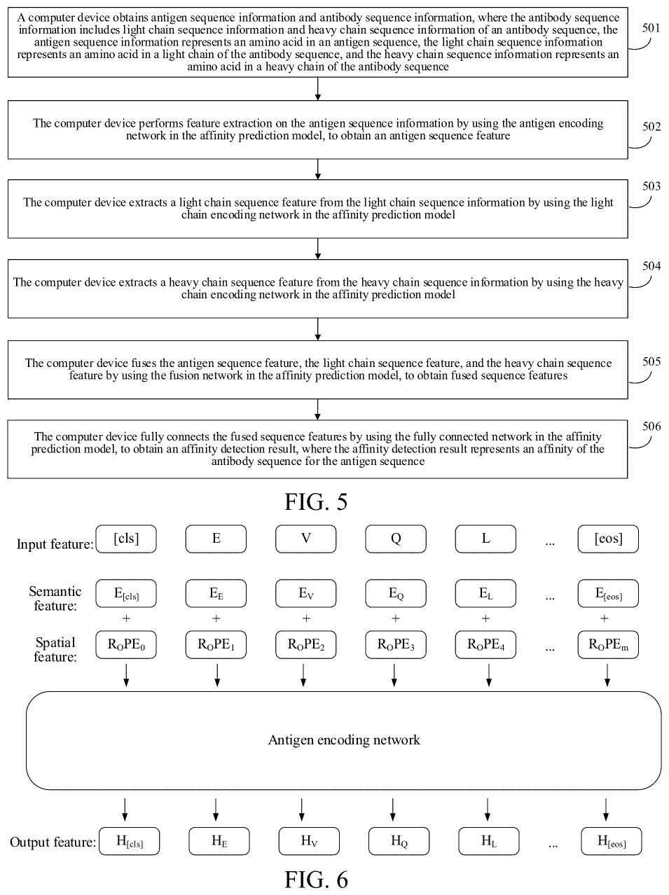

FIG. 5 a flowchart of a method for predicting an affinity of an antibody sequence for an antigen sequence according to some embodiments. Some embodiments is performed by a computer device. The computer device predicts an affinity detection result by using the affinity prediction model shown in FIG. 4. The affinity prediction model includes: an antigen encoding network, a light chain encoding network, a heavy chain encoding network, a fusion network, and a fully connected network. Referring to FIG. 5, the method includes the following operations.

501: The computer device obtains antigen sequence information and antibody sequence information, where the antibody sequence information includes light chain sequence information and heavy chain sequence information of an antibody sequence, the antigen sequence information represents an amino acid in an antigen sequence, the light chain sequence information represents an amino acid in a light chain of the antibody sequence, and the heavy chain sequence information represents an amino acid in a heavy chain of the antibody sequence.

The process of operation 501 is similar to the process of the foregoing operation 301.

502: The computer device performs feature extraction on the antigen sequence information by using the antigen encoding network in the affinity prediction model, to obtain an antigen sequence feature.

The computer device inputs the antigen sequence information into the antigen encoding network in the affinity prediction model, the antigen encoding network performs feature extraction on the antigen sequence information, and the antigen sequence feature is outputted.

In some embodiments, a network structure of the antigen encoding network may be a bidirectional encoder representations from transformers (BERT) model structure or a rotary transformers (Roformer) model structure. BERT is obtained by means of pretraining a deep bidirectional Transformer encoder. Roformer is a transformer with rotation position embedding.

In some embodiments, the process in which the computer device performs feature extraction on the antigen sequence information by using the antigen encoding network in the affinity prediction model, to obtain an antigen sequence feature includes: determining a first semantic feature and a first spatial feature of the antigen sequence information by using the antigen encoding network, and generating the antigen sequence feature according to the first semantic feature and the first spatial feature. The first semantic feature represents features of a plurality of amino acids in the antigen sequence information, and the first spatial feature represents features of positions of the plurality of amino acids in the antigen sequence information.

The antigen sequence information represents a plurality of amino acids in the antigen sequence. The antigen sequence information may include amino acid features of the plurality of amino acids in the antigen sequence. One amino acid feature may be referred to as one Token, and one Token represents one amino acid. A first Token in the antigen sequence information is [cls], and [cls] represents the first Token. A last Token in the antigen sequence information is [eos], and [eos] represents the last Token. The first semantic feature and the first spatial feature may be determined according to the plurality of amino acid features in the antigen sequence information. The first semantic feature represents features of the plurality of amino acids in the antigen sequence information. The first spatial feature represents features of positions of the plurality of amino acids in the antigen sequence information. The computer device generates, by using the antigen encoding network, the antigen sequence feature according to the first semantic feature and the first spatial feature.

In some embodiments, when the antigen sequence feature is extracted, the features of the amino acids in the antigen sequence and the features of the positions between the amino acids are comprehensively considered, so that features expressed by the extracted antigen sequence feature are more abundant, thereby improving accuracy of the antigen sequence feature, and further facilitating ensuring accuracy of subsequent affinity prediction based on the antigen sequence feature.

In some embodiments, the process of generating the antigen sequence feature according to the first semantic feature and the first spatial feature includes the following operations 5021 to 5023:

5021: Perform feature extraction on the first semantic feature and the first spatial feature, to obtain a first key feature, a first value feature, and a first query feature.

The first query feature, the first key feature, and the first value feature respectively belong to different feature spaces. Each amino acid in the antigen sequence has a respective first key feature, first value feature, and first query feature. The first value feature represents a feature of the amino acid. The first query feature and the first key feature are used for determining a feature vector of an attention weight.

In some embodiments, the process in which the computer device obtains a first query feature, a first key feature, and a first value feature includes: multiplying the first semantic feature by a first parameter matrix to obtain a semantic matrix, and obtaining, based on the semantic matrix, the first query feature, the first key feature, and the first value feature that correspond to the first semantic feature; and multiplying the first spatial feature by a second parameter matrix to obtain a spatial matrix, and obtaining, based on the spatial matrix, the first query feature, the first key feature, and the first value feature that correspond to the first spatial feature.

The first parameter matrix and the second parameter matrix are used for performing space transform. The first parameter matrix and the second parameter matrix are model parameters obtained through training. After obtaining the semantic matrix, the computer device splits the semantic matrix into the first query feature, the first key feature, and the first value feature that correspond to the first semantic feature. After the spatial matrix is obtained, the spatial matrix is split into the first query feature, the first key feature, and the first value feature that correspond to the first spatial feature. For example, using the first parameter matrix as an example, the first parameter matrix is a 3-dimensional parameter matrix. The semantic matrix obtained by multiplying the first semantic feature by the first parameter matrix is a 3-dimensional semantic matrix. The computer device separately uses each dimension of the semantic matrix as the first query feature, the first key feature, and the first value feature that correspond to the first semantic feature.

5022: Fuse the first key feature, the first value feature, and the first query feature, to obtain a candidate antigen sequence feature.

After obtaining the first key feature, the first value feature, and the first query feature, the computer device fuses the first key feature, the first value feature, and the first query feature, to obtain a candidate antigen sequence feature.

In some embodiments, the process in which the computer device fuses the first key feature, the first value feature, and the first query feature, to obtain a candidate antigen sequence feature includes: normalizing a product of the first query feature, the first key feature, and a scaling factor, to obtain a normalized feature, and determining a product of the normalized feature and the first value feature as the candidate antigen sequence feature. The computer device obtains the scaling factor. The scaling factor represents a normalization scaling multiple. In some embodiments, the computer device normalizes the product by using the scaling factor as the normalization parameter, to obtain the normalized feature. The normalized feature represents a correlation between the first query feature and the first key feature. Then, the computer device may use the normalized feature as a weight of the first value feature. Therefore, the computer device determines the product of the normalized feature and the first value feature as the candidate antigen sequence feature.

The computer device fuses the first key feature, the first value feature, and the first query feature, to obtain the candidate antigen sequence feature by using the following formula:

$$A = softmax ( \frac{{QK}^{T}}{\sqrt{{d}_{k}}} ) V ,$$

where A represents the candidate antigen sequence feature, Q represents the first query feature, K represents the first key feature, T represents transposition,

$$\frac{1}{\sqrt{{d}_{k}}}$$

represents the scaling factor, V represents the first value feature, and softmax(·) represents a normalization function.

5023: Convert the candidate antigen sequence feature repeatedly, to obtain the antigen sequence feature.

The affinity prediction model may include a layer structure for performing repeated conversion on the candidate antigen sequence feature. The computer device may input the candidate antigen sequence feature to a first layer of the layer structure, output a feature extraction result to a next layer after feature extraction is performed on each layer, and continue to perform feature extraction until a last layer of the layer structure outputs the antigen sequence feature. In other embodiments, feature extraction may be performed by combining feature extraction results respectively outputted by at least two previous layers starting from a third layer in the structure.

In some embodiments, the computer device converts the candidate antigen sequence feature repeatedly by using a feedforward neural network, to obtain the antigen sequence feature. The feedforward neural network may be represented by using the following formula:

$$B = F F N ( A ) = max ( 0 , {AW}_{1} + {b}_{1} ) {W}_{2} + {b}_{2} ,$$

where B represents the antigen sequence feature, FFN(·) represents the feedforward neural network, A represents the candidate antigen sequence feature, max(·) represents maximizing, and W₁, b₁, W₂, and b₂ represent model parameters in the feedforward neural network.

FIG. 6 is a schematic diagram of extraction of an antigen sequence feature according to some embodiments. As shown in FIG. 6, an input feature is a Token in the antigen sequence information. For example, the Token in the antigen sequence information includes [cls], E, V, Q, L, . . . , [eos]. A semantic feature and a spatial feature of each amino acid are determined according to the Token in the antigen sequence information. Feature extraction is performed on the semantic feature and the spatial feature of each amino acid by using the antigen encoding network to obtain an output feature. The output feature is an antigen sequence feature. A dimension of the antigen sequence feature is the same as a dimension of the antigen sequence information. The antigen sequence feature includes a deep feature of each amino acid in the antigen sequence.

503: The computer device extracts a light chain sequence feature from the light chain sequence information by using the light chain encoding network in the affinity prediction model.

The computer device inputs the light chain sequence information into the light chain encoding network in the affinity prediction model, the light chain encoding network performs feature extraction on the light chain sequence information, and the light chain sequence feature is outputted.

In some embodiments, a network structure of the light chain encoding network may be a BERT model structure or a Roformer model structure. BERT is obtained by means of pretraining a deep bidirectional Transformer encoder. Roformer is a transformer with rotation position embedding.

In some embodiments, the process in which the computer device extracts a light chain sequence feature from the light chain sequence information by using the light chain encoding network in the affinity prediction model includes: determining a second semantic feature and a second spatial feature of the light chain sequence information by using the light chain encoding network, and generating the light chain sequence feature according to the second semantic feature and the second spatial feature. The second semantic feature represents features of a plurality of amino acids in the light chain sequence information, and the second spatial feature represents features of positions of the plurality of amino acids in the light chain sequence information.

The light chain sequence information represents a plurality of amino acids in the light chain sequence. The light chain sequence information may include amino acid features of the plurality of amino acids in the light chain sequence. One amino acid feature may be referred to as one Token, and one Token represents one amino acid. A first Token in the light chain sequence information is [cls], and [cls] represents the first Token. A last Token in the light chain sequence information is [eos], and [eos] represents the last Token. The second semantic feature and the second spatial feature may be determined according to the plurality of amino acid features in the light chain sequence information. The second semantic feature represents features of the plurality of amino acids in the light chain sequence information. The second spatial feature represents features of positions of the plurality of amino acids in the light chain sequence information. The computer device generates, by using the light chain encoding network, the light chain sequence feature according to the second semantic feature and the second spatial feature.

In some embodiments, when the light chain sequence feature is extracted, the features of the amino acids in the light chain sequence and the features of the positions between the amino acids are comprehensively considered, so that features expressed by the extracted light chain sequence feature are more abundant, thereby improving accuracy of the light chain sequence feature, and further facilitating ensuring accuracy of subsequent affinity prediction based on the light chain sequence feature.

In some embodiments, the process of generating the light chain sequence feature according to the second semantic feature and the second spatial feature includes the following operations 5031 to 5033:

5031: Perform feature extraction on the second semantic feature and the second spatial feature, to obtain a second key feature, a second value feature, and a second query feature.

Each amino acid in the light chain has a respective second key feature, second value feature, and second query feature. The second value feature represents a feature of the amino acid. The second query feature and the second key feature are used for determining a feature vector of an attention weight.

In some embodiments, the process in which the computer device obtains a second query feature, a second key feature, and a second value feature includes: multiplying the second semantic feature by a third parameter matrix to obtain a semantic matrix, and obtaining, based on the semantic matrix, the second query feature, the second key feature, and the second value feature that correspond to the second semantic feature; and multiplying the second spatial feature by a fourth parameter matrix to obtain a spatial matrix, and obtaining, based on the spatial matrix, the second query feature, the second key feature, and the second value feature that correspond to the second spatial feature.

The third parameter matrix and the fourth parameter matrix are used for performing space transform. The third parameter matrix and the fourth parameter matrix are model parameters obtained through training.

5032: Fuse the second key feature, the second value feature, and the second query feature, to obtain a candidate light chain sequence feature.

After obtaining the second key feature, the second value feature, and the second query feature, the computer device fuses the second key feature, the second value feature, and the second query feature, to obtain a candidate light chain sequence feature.

In some embodiments, the process in which the computer device fuses the second key feature, the second value feature, and the second query feature, to obtain a candidate light chain sequence feature includes: normalizing a product of the second query feature, the second key feature, and a scaling factor, to obtain a normalized feature, and determining a product of the normalized feature and the second value feature as the candidate light chain sequence feature.

5033: Convert the candidate light chain sequence feature repeatedly, to obtain the light chain sequence feature.

The affinity prediction model may include a layer structure for performing repeated conversion on the candidate light chain sequence feature. The computer device may input the candidate light chain sequence feature to a first layer of the layer structure, output a feature extraction result to a next layer after feature extraction is performed on each layer, and continue to perform feature extraction until a last layer of the layer structure outputs the antigen sequence feature. In other embodiments, feature extraction may be performed by combining feature extraction results respectively outputted by at least two previous layers starting from a third layer in the structure.

In some embodiments, the computer device converts the candidate light chain sequence feature repeatedly by using a feedforward neural network, to obtain the light chain sequence feature.

504: The computer device extracts a heavy chain sequence feature from the heavy chain sequence information by using the heavy chain encoding network in the affinity prediction model.

The computer device inputs the heavy chain sequence information into the heavy chain encoding network in the affinity prediction model, the heavy chain encoding network performs feature extraction on the heavy chain sequence information, and the heavy chain sequence feature is outputted.

In some embodiments, a network structure of the heavy chain encoding network may be a BERT model structure or a Roformer model structure. BERT is obtained by means of pretraining a deep bidirectional Transformer encoder. Roformer is a transformer with rotation position embedding.

In some embodiments, the process in which the computer device extracts a heavy chain sequence feature from the heavy chain sequence information by using the heavy chain encoding network in the affinity prediction model includes: determining a third semantic feature and a third spatial feature of the heavy chain sequence information by using the heavy chain encoding network, and generating the heavy chain sequence feature according to the third semantic feature and the third spatial feature. The third semantic feature represents features of a plurality of amino acids in the heavy chain sequence information, and the third spatial feature represents features of positions of the plurality of amino acids in the heavy chain sequence information.

The heavy chain sequence information represents a plurality of amino acids in the heavy chain sequence. The heavy chain sequence information may include amino acid features of the plurality of amino acids in the heavy chain sequence. One amino acid feature may be referred to as one Token, and one Token represents one amino acid. A first Token in the heavy chain sequence information is [cls], and [cls] represents the first Token. A last Token in the heavy chain sequence information is [eos], and [eos] represents the last Token. The third semantic feature and the third spatial feature may be determined according to the plurality of amino acid features in the heavy chain sequence information. The third semantic feature represents features of the plurality of amino acids in the heavy chain sequence information. The third spatial feature represents features of positions of the plurality of amino acids in the heavy chain sequence information. The computer device generates, by using the heavy chain encoding network, the heavy chain sequence feature according to the third semantic feature and the third spatial feature.

In some embodiments, when the heavy chain sequence feature is extracted, the features of the amino acids in the heavy chain sequence and the features of the positions between the amino acids are comprehensively considered, so that features expressed by the extracted heavy chain sequence feature are more abundant, thereby improving accuracy of the heavy chain sequence feature, and further facilitating ensuring accuracy of subsequent affinity prediction based on the heavy chain sequence feature.

In some embodiments, the process of generating the heavy chain sequence feature according to the third semantic feature and the third spatial feature includes the following operations 5041 to 5043:

5041: Perform feature extraction on the third semantic feature and the third spatial feature, to obtain a third key feature, a third value feature, and a third query feature.

Each amino acid in the heavy chain has a respective third key feature, third value feature, and third query feature. The third value feature represents a feature of the amino acid. The third query feature and the third key feature are used for determining a feature vector of an attention weight.

In some embodiments, the process in which the computer device obtains a third query feature, a third key feature, and a third value feature includes: multiplying the third semantic feature by a fifth parameter matrix to obtain a semantic matrix, and obtaining, based on the semantic matrix, the third query feature, the third key feature, and the third value feature that correspond to the third semantic feature; and multiplying the third spatial feature by a sixth parameter matrix to obtain a spatial matrix, and obtaining, based on the spatial matrix, the third query feature, the third key feature, and the third value feature that correspond to the third spatial feature.

The fifth parameter matrix and the sixth parameter matrix are used for performing space transform. The fifth parameter matrix and the sixth parameter matrix are model parameters obtained through training.

5042: Fuse the third key feature, the third value feature, and the third query feature, to obtain a candidate heavy chain sequence feature.

After obtaining the third key feature, the third value feature, and the third query feature, the computer device fuses the third key feature, the third value feature, and the third query feature, to obtain a candidate heavy chain sequence feature.

In some embodiments, the process in which the computer device fuses the third key feature, the third value feature, and the third query feature, to obtain a candidate heavy chain sequence feature includes: normalizing a product of the third query feature, the third key feature, and a scaling factor, to obtain a normalized feature, and determining a product of the normalized feature and the third value feature as the candidate heavy chain sequence feature.

5043: Convert the candidate heavy chain sequence feature repeatedly, to obtain the heavy chain sequence feature.

The affinity prediction model may include a layer structure for performing repeated conversion on the candidate heavy chain sequence feature. The computer device may input the candidate heavy chain sequence feature to a first layer of the layer structure, output a feature extraction result to a next layer after feature extraction is performed on each layer, and continue to perform feature extraction until a last layer of the layer structure outputs the antigen sequence feature. In other embodiments, feature extraction may be performed by combining feature extraction results respectively outputted by at least two previous layers starting from a third layer in the structure.

In some embodiments, the computer device converts the candidate heavy chain sequence feature repeatedly by using a feedforward neural network, to obtain the heavy chain sequence feature.

505: The computer device fuses the antigen sequence feature, the light chain sequence feature, and the heavy chain sequence feature by using the fusion network in the affinity prediction model, to obtain fused sequence features.

In some embodiments, the fusion network includes a first convolutional network, a second convolutional network, and a third convolutional network. The computer device inputs the antigen sequence feature into the first convolutional network. The first convolutional network convolves the antigen sequence feature repeatedly, to obtain a deep antigen sequence feature. The light chain sequence feature is inputted into the second convolutional network. The second convolutional network convolves the light chain sequence feature repeatedly, to obtain a deep light chain sequence feature. The heavy chain sequence feature is inputted into the third convolutional network. The third convolutional network convolves the heavy chain sequence feature repeatedly, to obtain a deep heavy chain sequence feature. The deep antigen sequence feature, the deep light chain sequence feature, and the deep heavy chain sequence feature are fused (for example, concatenated), to obtain fused sequence features.

In some embodiments, network structures of the first convolutional network, the second convolutional network, and the third convolutional network are an MF-CNN model structure.

The MF-CNN model structure may be represented by the following formula:

M=MultiConv(Roformer(seq)),

where M represents an output of an MF-CNN model (i.e. a deep sequence feature in this application), Roformer(seq) represents an output of an encoding network (i.e. a sequence feature in this application), and MultiConv(·) represents the MF-CNN model.

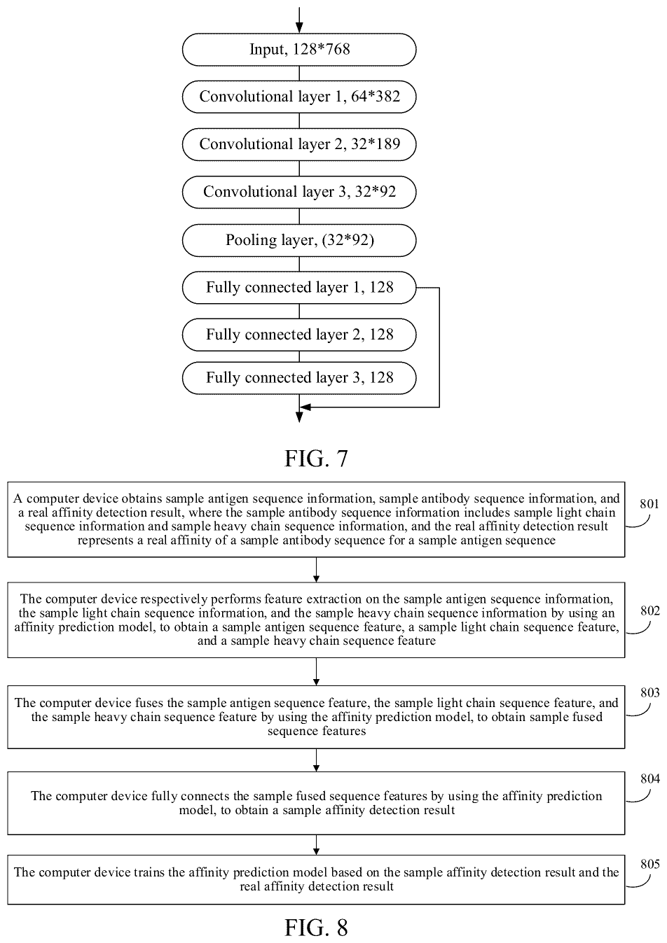

For the MF-CNN model structure, refer to FIG. 7. The MF-CNN model includes a plurality of convolutional neural networks (CNN). Each CNN includes three convolutional layers, one pooling layer, and three fully connected layers.

In some embodiments, the network structures of the first convolutional network, the second convolutional network, and the third convolutional network may be a recurrent neural network having time sequence information.

506: The computer device fully connects the fused sequence features by using the fully connected network in the affinity prediction model, to obtain an affinity detection result, where the affinity detection result represents an affinity of the antibody sequence for the antigen sequence.

The computer device inputs the fused sequence features into the fully connected network. The fully connected network fully connects the fused sequence features, to output an affinity detection result.

In some embodiments, the affinity detection result is an affinity value. A larger affinity value indicates a greater affinity of the antibody sequence for the antigen sequence. A smaller affinity value indicates a smaller affinity of the antibody sequence for the antigen sequence. In some embodiments, the affinity detection result is a binary classification result. In a case that the affinity detection result is a first value, it indicates that the antibody sequence has an affinity for the antigen sequence. In a case that the affinity detection result is a second value, it indicates that the antibody sequence has no affinity for the antigen sequence. For example, the first value is 1, and the second value is 0.

In some embodiments, the fully connected network is a deep neural network (DNN). In some embodiments, the fully connected network may be represented by using the following formula:

$$Out = FC ( Concat [ M ] ) + Concat [ M ] ,$$

where M represents the fused sequence feature, Out represents the affinity detection result, Concat represents a connection function, and FC(·) represents the fully connected layer.

In the method according to some embodiments, when an affinity of an antibody sequence for an antigen sequence is predicted, a feature of an amino acid in the antigen sequence, a feature of an amino acid in a light chain of the antibody sequence, and a feature of an amino acid in a heavy chain of the antibody sequence are comprehensively considered. In terms of the heavy chain and the light chain, potential associations of the feature of the amino acid in the antigen sequence with the feature of the amino acid in the light chain and the feature of the amino acid in the heavy chain are considered, so as to predict the affinity of the antibody sequence for the antigen sequence. Not only factors are considered comprehensively, but also two granularities of the heavy chain and the light chain are divided, which is beneficial to improving the accuracy of affinity prediction.

FIG. 8 is a flowchart of a training method of an affinity prediction model according to some embodiments. Some embodiments is performed by a computer device. Referring to FIG. 8, the method includes the following operations.

801: The computer device obtains sample antigen sequence information, sample antibody sequence information, and a real affinity detection result, where the sample antibody sequence information includes sample light chain sequence information and sample heavy chain sequence information, and the real affinity detection result represents a real affinity of a sample antibody sequence for a sample antigen sequence.

The sample antibody sequence information includes sample light chain sequence information and sample heavy chain sequence information of the sample antibody sequence. The sample antigen sequence information represents an amino acid in the sample antigen sequence. The sample light chain sequence information represents an amino acid in a light chain of the sample antibody sequence. The sample heavy chain sequence information represents an amino acid in a heavy chain of the sample antibody sequence.

The process of operation 801 is similar to the process of the foregoing operation 301.

802: The computer device respectively performs feature extraction on the sample antigen sequence information, the sample light chain sequence information, and the sample heavy chain sequence information by using an affinity prediction model, to obtain a sample antigen sequence feature, a sample light chain sequence feature, and a sample heavy chain sequence feature.

The computer device inputs the sample antigen sequence information, the sample light chain sequence information, and the sample heavy chain sequence information into the affinity prediction model. The affinity prediction model respectively performs feature extraction on the sample antigen sequence information, the sample light chain sequence information, and the sample heavy chain sequence information, to obtain the sample antigen sequence feature, the sample light chain sequence feature, and the sample heavy chain sequence feature.

In some embodiments, the affinity prediction model includes an antigen encoding network, a light chain encoding network, and a heavy chain encoding network. The process in which the computer device obtains a sample antigen sequence feature, a sample light chain sequence feature, and a sample heavy chain sequence feature includes the following operations 8021 to 8023.

8021: Perform feature extraction on the sample antigen sequence information by using the antigen encoding network, to obtain the sample antigen sequence feature.

In some embodiments, the computer device determines a first sample semantic feature and a first sample spatial feature of the sample antigen sequence information by using the antigen encoding network, and performs feature extraction on the first sample semantic feature and the first sample spatial feature, to obtain the sample antigen sequence feature. The first sample semantic feature represents features of a plurality of amino acids in the sample antigen sequence information, and the first sample spatial feature represents features of positions of the plurality of amino acids in the sample antigen sequence information.

In some embodiments, the process of performing feature extraction on the first sample semantic feature and the first sample spatial feature, to obtain the sample antigen sequence feature includes: performing feature extraction on the first sample semantic feature and the first sample spatial feature, to obtain a first sample key feature, a first sample value feature, and a first sample query feature; fusing the first sample key feature, the first sample value feature, and the first sample query feature, to obtain a candidate sample antigen sequence feature; and convert the candidate sample antigen sequence feature repeatedly, to obtain the sample antigen sequence feature.

8022: Perform feature extraction on the sample light chain sequence information by using the light chain encoding network, to obtain the sample light chain sequence feature.

In some embodiments, the computer device determines a second sample semantic feature and a second sample spatial feature of the sample light chain sequence information by using the light chain encoding network, and performs feature extraction on the second sample semantic feature and the second sample spatial feature, to obtain the sample light chain sequence feature. The second sample semantic feature represents features of a plurality of amino acids in the sample light chain sequence information, and the second sample spatial feature represents features of positions of the plurality of amino acids in the sample light chain sequence information.

In some embodiments, the process of performing feature extraction on the second sample semantic feature and the second sample spatial feature, to obtain the sample light chain sequence feature includes: performing feature extraction on the second sample semantic feature and the second sample spatial feature, to obtain a second sample key feature, a second sample value feature, and a second sample query feature; fusing the second sample key feature, the second sample value feature, and the second sample query feature, to obtain a candidate sample light chain sequence feature; and convert the candidate sample light chain sequence feature repeatedly, to obtain the sample light chain sequence feature.

8023: Perform feature extraction on the sample heavy chain sequence information by using the heavy chain encoding network, to obtain the sample heavy chain sequence feature.

In some embodiments, the computer device determines a third sample semantic feature and a third sample spatial feature of the sample heavy chain sequence information by using the heavy chain encoding network, and performs feature extraction on the third sample semantic feature and the third sample spatial feature, to obtain the sample heavy chain sequence feature. The third sample semantic feature represents features of a plurality of amino acids in the sample heavy chain sequence information, and the third sample spatial feature represents features of positions of the plurality of amino acids in the sample heavy chain sequence information.

In some embodiments, the process of performing feature extraction on the third sample semantic feature and the third sample spatial feature, to obtain the sample heavy chain sequence feature includes: performing feature extraction on the third sample semantic feature and the third sample spatial feature, to obtain a third sample key feature, a third sample value feature, and a third sample query feature; fuse the third sample key feature, the third sample value feature, and the third sample query feature, to obtain a sample candidate heavy chain sequence feature; and convert the sample candidate heavy chain sequence feature repeatedly, to obtain the sample heavy chain sequence feature.

803: The computer device fuses the sample antigen sequence feature, the sample light chain sequence feature, and the sample heavy chain sequence feature by using the affinity prediction model, to obtain sample fused sequence features.

In some embodiments, the affinity prediction model includes a fusion network. The computer device fuses the sample antigen sequence feature, the sample light chain sequence feature, and the sample heavy chain sequence feature by using the fusion network, to obtain sample fused sequence features.

In some embodiments, the fusion network includes a first convolutional network, a second convolutional network, and a third convolutional network. The computer device inputs the sample antigen sequence feature into the first convolutional network. The first convolutional network convolves the sample antigen sequence feature repeatedly, to obtain a deep sample antigen sequence feature. The sample light chain sequence feature is inputted into the second convolutional network. The second convolutional network convolves the sample light chain sequence feature repeatedly, to obtain a deep sample light chain sequence feature. The sample heavy chain sequence feature is inputted into the third convolutional network. The third convolutional network convolves the sample heavy chain sequence feature repeatedly, to obtain a deep sample heavy chain sequence feature. The deep sample antigen sequence feature, the deep sample light chain sequence feature, and the deep sample heavy chain sequence feature are fused, to obtain sample fused sequence features.

804: The computer device fully connects the sample fused sequence features by using the affinity prediction model, to obtain a sample affinity detection result.

In some embodiments, the affinity prediction model includes a fully connected network. The computer device fully connects the sample fused sequence features by using the fully connected network, to obtain the sample affinity detection result.

805: The computer device trains the affinity prediction model based on the sample affinity detection result and the real affinity detection result.

The computer device obtains a real affinity detection result between the sample antigen sequence and the sample antibody sequence. The real affinity detection result represents a real affinity of the sample antibody sequence for the sample antigen sequence. The sample affinity detection result obtained by the computer device is an affinity predicted by the affinity prediction model. The computer device trains the affinity prediction model based on a difference between the sample affinity detection result and the real affinity detection result.

Because an objective of the affinity prediction model is to predict the real affinity of the sample antibody sequence for the sample antigen sequence, a higher similarity between the sample affinity detection result and the real affinity detection result indicates a more accurate affinity prediction model. The computer device trains the affinity prediction model according to the difference between the sample affinity detection result and the real affinity detection result, so that the difference between the sample affinity detection result obtained by using the trained affinity prediction model and the real affinity detection result is reduced, to improve a prediction capability of the affinity prediction model, thereby improving accuracy of the affinity prediction model.

In some embodiments, the computer device repeatedly performs the foregoing operations 801 to 805, iteratively trains the affinity prediction model, and stops training the affinity prediction model in response to a quantity of iterations reaching a first threshold. The computer device stops training the affinity prediction model in response to a loss value obtained in a current iteration is not greater than a second threshold. The first threshold and the second threshold are any values. For example, the first threshold is 1000, 1500, or the like, and the second threshold is 0.004, 0.003, or the like.

In the method according to some embodiments, when an affinity prediction model for predicting an affinity of an antibody sequence for an antigen sequence is trained, a feature of an amino acid in the antigen sequence, a feature of an amino acid in a light chain of the antibody sequence, and a feature of an amino acid in a heavy chain of the antibody sequence are comprehensively considered. In terms of the heavy chain and the light chain, the affinity prediction model learns potential associations of the feature of the amino acid in the antigen sequence with the feature of the amino acid in the light chain and the feature of the amino acid in the heavy chain, so as to predict the affinity of the antibody sequence for the antigen sequence. Not only factors are considered comprehensively, but also two granularities of the heavy chain and the light chain are divided, which is beneficial to improving a prediction capability of the affinity prediction model.

The foregoing affinity prediction model includes an antigen encoding network, a light chain encoding network, and a heavy chain encoding network. Before the affinity prediction model is trained, the antigen encoding network, the light chain encoding network, and the heavy chain encoding network may be untrained encoding networks, or may be encoding networks that have been pretrained. After the pretraining is completed, the affinity prediction model is constructed by using the antigen encoding network, the light chain encoding network, and the heavy chain encoding network that are obtained through pretraining. Then the affinity prediction model is trained.

Pretraining refers to a process of performing unsupervised learning by using a large-scale data set. Training is performed on a large amount of unlabeled data, to automatically learn patterns and rules in the data, thereby better processing various tasks. Pretraining is usually performed by using a neural network, and a high-level feature representation of the data is extracted in a layer-by-layer learning mode.

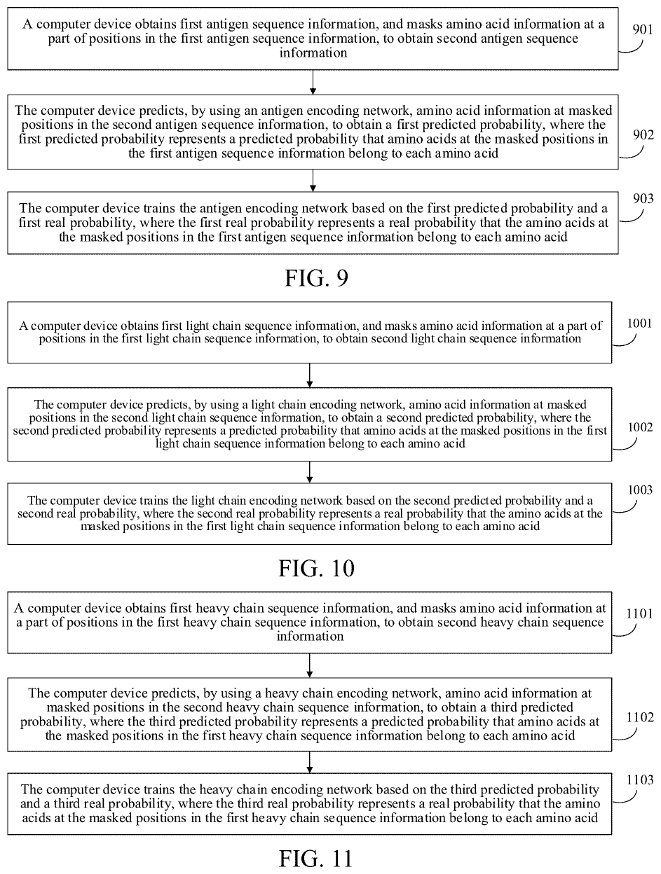

For the pretraining process of the antigen encoding network, the light chain encoding network, and the heavy chain encoding network, refer to the following embodiments shown in FIG. 9 to FIG. 11.

FIG. 9 is a flowchart of a pretraining method of an antigen encoding network according to some embodiments. Some embodiments is performed by a computer device. Referring to FIG. 9, the method includes the following operations.

901: The computer device obtains first antigen sequence information, and masks amino acid information at a part of positions in the first antigen sequence information, to obtain second antigen sequence information.

The first antigen sequence information may be antigen sequence information of any antigen sequence. The first antigen sequence information represents an amino acid in the antigen sequence. The antigen sequence includes a plurality of amino acids. The first antigen sequence information includes amino acid information at a plurality of positions. The amino acid information at each position represents an amino acid at this position.

The computer device randomly masks amino acid information at a part of positions in the first antigen sequence information, to obtain second antigen sequence information. Amino acid information at a part of positions in the second antigen sequence information is unknown.

In some embodiments, the computer device replaces the amino acid information at a part of positions in the first antigen sequence information with a preset character. For example, the preset character is [Mask]. For example, the first antigen sequence information is “MDVLY”, “V” and “Y” in the first antigen sequence information are masked. To be specific, “V” and “Y” are replaced with [Mask]. The obtained second antigen sequence information is “M D [Mask] L [Mask]”.

902: The computer device predicts, by using an antigen encoding network, amino acid information at masked positions in the second antigen sequence information, to obtain a first predicted probability, where the first predicted probability represents a predicted probability that amino acids at the masked positions in the first antigen sequence information belong to each amino acid.

The computer device inputs the second antigen sequence information into the antigen encoding network. The antigen encoding network predicts the amino acid information at masked positions in the second antigen sequence information, to output the first predicted probability.

In some embodiments, in a pretraining process, compared with the affinity prediction model, the antigen encoding network further includes an output layer. After the antigen encoding network is pretrained, the output layer is removed from the antigen encoding network, and the antigen encoding network from which the output layer is removed is used for constructing the affinity prediction model. Then the computer device determines a semantic feature and a spatial feature of the second antigen sequence information by using the antigen encoding network, and performs feature extraction on the semantic feature and the spatial feature, to obtain an antigen sequence feature. The semantic feature represents features of a plurality of amino acids in the antigen sequence information, and the spatial feature represents features of positions of the plurality of amino acids in the antigen sequence information. Then, the output layer is used for predicting the antigen sequence feature, to obtain the first predicted probability.

The process in which the antigen encoding network extracts the antigen sequence feature of the second antigen sequence information is the same as the process of extracting the antigen sequence feature in some embodiments shown in FIG. 5.

903: The computer device trains the antigen encoding network based on the first predicted probability and a first real probability, where the first real probability represents a real probability that the amino acids at the masked positions in the first antigen sequence information belong to each amino acid.

The computer device obtains a first real probability. The first real probability represents a real probability that the amino acids at the masked positions in the first antigen sequence information belong to each amino acid. The first predicted probability obtained by the computer device is predicted by the antigen encoding network. The computer device trains the antigen encoding network based on a difference between the first predicted probability and the first real probability.

Because an objective of the antigen encoding network is to predict a real probability that the amino acids at the masked positions in the first antigen sequence information belong to each amino acid. Therefore, a higher similarity between the first predicted probability and the first real probability indicates a higher capability of extracting the antigen sequence feature by the antigen encoding network. The computer device trains the first predicted probability and the first real probability according to the difference between the first predicted probability and the first real probability, so that the difference between the first predicted probability obtained by using the trained antigen encoding network and the first real probability is reduced, to improve a feature extraction capability of the antigen encoding network. Therefore, the antigen encoding network initially learns to extract the antigen sequence feature.

In the method according to some embodiments, an antigen encoding network is pretrained in a self-supervised mode according to sample antigen sequence information, thereby improving a feature extraction capability of the antigen encoding network for antigen sequence information. Subsequently, an affinity prediction model is constructed by using the pretrained antigen encoding network, thereby helping to reduce a training pressure of the affinity prediction model, and ensuring accuracy of the affinity prediction model.

FIG. 10 is a flowchart of a pretraining method of a light chain encoding network according to some embodiments. Some embodiments is performed by a computer device. Referring to FIG. 10, the method includes the following operations.

1001: The computer device obtains first light chain sequence information, and masks amino acid information at a part of positions in the first light chain sequence information, to obtain second light chain sequence information.

The first light chain sequence information may be light chain sequence information of any light chain. The first light chain sequence information represents an amino acid in the light chain. The light chain includes a plurality of amino acids. The first light chain sequence information includes amino acid information at a plurality of positions. The amino acid information at each position represents an amino acid at this position.

The computer device randomly masks amino acid information at a part of positions in the first light chain sequence information, to obtain second light chain sequence information. Amino acid information at a part of positions in the second light chain sequence information is unknown. A masking mode of the first light chain sequence information is the same as the masking mode of the first antigen sequence information in some embodiments shown in FIG. 9.

1002: The computer device predicts, by using a light chain encoding network, amino acid information at masked positions in the second light chain sequence information, to obtain a second predicted probability, where the second predicted probability represents a predicted probability that amino acids at the masked positions in the first light chain sequence information belong to each amino acid.

The computer device inputs the second light chain sequence information into the light chain encoding network. The light chain encoding network predicts the amino acid information at masked positions in the second light chain sequence information, to output the second predicted probability.

In some embodiments, in a pretraining process, compared with the affinity prediction model, the light chain encoding network further includes an output layer. After the light chain encoding network is pretrained, the output layer is removed from the light chain encoding network, and the light chain encoding network from which the output layer is removed is used for constructing the affinity prediction model. Then the computer device determines a semantic feature and a spatial feature of the second light chain sequence information by using the light chain encoding network, and performs feature extraction on the semantic feature and the spatial feature, to obtain a light chain sequence feature. The semantic feature represents features of a plurality of amino acids in the light chain sequence information, and the spatial feature represents features of positions of the plurality of amino acids in the light chain sequence information. Then, the output layer is used for predicting the light chain sequence feature, to obtain the second predicted probability.

The process in which the light chain encoding network extracts the light chain sequence feature of the second light chain sequence information is the same as the process of extracting the light chain sequence feature in some embodiments shown in FIG. 5.

1003: The computer device trains the light chain encoding network based on the second predicted probability and a second real probability, where the second real probability represents a real probability that the amino acids at the masked positions in the first light chain sequence information belong to each amino acid.

The computer device obtains a second real probability. The second real probability represents a real probability that the amino acids at the masked positions in the first light chain sequence information belong to each amino acid. The second predicted probability obtained by the computer device is predicted by the light chain encoding network. The computer device trains the light chain encoding network based on a difference between the second predicted probability and the second real probability.

Because an objective of the light chain encoding network is to predict a real probability that the amino acids at the masked positions in the first light chain sequence information belong to each amino acid. Therefore, a higher similarity between the second predicted probability and the second real probability indicates a higher capability of extracting the light chain sequence feature by the light chain encoding network. The computer device trains the second predicted probability and the second real probability according to the difference between the second predicted probability and the second real probability, so that the difference between the second predicted probability obtained by using the trained light chain encoding network and the second real probability is reduced, to improve a feature extraction capability of the light chain encoding network. Therefore, the light chain encoding network initially learns to extract the light chain sequence feature.

In the method according to some embodiments, a light chain encoding network is pretrained in a self-supervised mode according to sample light chain sequence information, thereby improving a feature extraction capability of the light chain encoding network for light chain sequence information. Subsequently, an affinity prediction model is constructed by using the pretrained light chain encoding network, thereby helping to reduce a training pressure of the affinity prediction model, and ensuring accuracy of the affinity prediction model.

FIG. 11 is a flowchart of a pretraining method of a light chain encoding network according to some embodiments. Some embodiments is performed by a computer device. Referring to FIG. 11, the method includes the following operations.

1101: The computer device obtains first heavy chain sequence information, and masks amino acid information at a part of positions in the first heavy chain sequence information, to obtain second heavy chain sequence information.

The first heavy chain sequence information may be heavy chain sequence information of any heavy chain. The first heavy chain sequence information represents an amino acid in the heavy chain. The heavy chain includes a plurality of amino acids. The first heavy chain sequence information includes amino acid information at a plurality of positions. The amino acid information at each position represents an amino acid at this position.

The computer device randomly masks amino acid information at a part of positions in the first heavy chain sequence information, to obtain second heavy chain sequence information. Amino acid information at a part of positions in the second heavy chain sequence information is unknown. A masking mode of the first heavy chain sequence information is the same as the masking mode of the first antigen sequence information in some embodiments shown in FIG. 9.

1102: The computer device predicts, by using a heavy chain encoding network, amino acid information at masked positions in the second heavy chain sequence information, to obtain a third predicted probability, where the third predicted probability represents a predicted probability that amino acids at the masked positions in the first heavy chain sequence information belong to each amino acid.

The computer device inputs the second heavy chain sequence information into the heavy chain encoding network. The heavy chain encoding network predicts the amino acid information at masked positions in the second heavy chain sequence information, to output the third predicted probability.

In some embodiments, in a pretraining process, compared with the affinity prediction model, the heavy chain encoding network further includes an output layer. After the heavy chain encoding network is pretrained, the output layer is removed from the heavy chain encoding network, and the heavy chain encoding network from which the output layer is removed is used for constructing the affinity prediction model. Then the computer device determines a semantic feature and a spatial feature of the second heavy chain sequence information by using the heavy chain encoding network, and performs feature extraction on the semantic feature and the spatial feature, to obtain a heavy chain sequence feature. The semantic feature represents features of a plurality of amino acids in the heavy chain sequence information, and the spatial feature represents features of positions of the plurality of amino acids in the heavy chain sequence information. Then, the output layer is used for predicting the heavy chain sequence feature, to obtain the third predicted probability.

The process in which the heavy chain encoding network extracts the heavy chain sequence feature of the second heavy chain sequence information is the same as the process of extracting the heavy chain sequence feature in some embodiments shown in FIG. 5.

1103: The computer device trains the heavy chain encoding network based on the third predicted probability and a third real probability, where the third real probability represents a real probability that the amino acids at the masked positions in the first heavy chain sequence information belong to each amino acid.

The computer device obtains a third real probability. The third real probability represents a real probability that the amino acids at the masked positions in the first heavy chain sequence information belong to each amino acid. The third predicted probability obtained by the computer device is predicted by the heavy chain encoding network. The computer device trains the heavy chain encoding network based on a difference between the third predicted probability and the third real probability.

Because an objective of the heavy chain encoding network is to predict a real probability that the amino acids at the masked positions in the first heavy chain sequence information belong to each amino acid. Therefore, a higher similarity between the third predicted probability and the third real probability indicates a higher capability of extracting the heavy chain sequence feature by the heavy chain encoding network. The computer device trains the third predicted probability and the third real probability according to the difference between the third predicted probability and the third real probability, so that the difference between the third predicted probability obtained by using the trained heavy chain encoding network and the third real probability is reduced, to improve a feature extraction capability of the heavy chain encoding network. Therefore, the heavy chain encoding network initially learns to extract the heavy chain sequence feature.

In the method according to some embodiments, a heavy chain encoding network is pretrained in a self-supervised mode according to sample heavy chain sequence information, thereby improving a feature extraction capability of the heavy chain encoding network for heavy chain sequence information. Subsequently, an affinity prediction model is constructed by using the pretrained heavy chain encoding network, thereby helping to reduce a training pressure of the affinity prediction model, and ensuring accuracy of the affinity prediction model.

To verify the effectiveness of some embodiments, the method according to some embodiments is used for performing a performance test on a plurality of data sets of antigen sequence-antibody sequence, including a binary classification data set and an affinity regression data set. An affinity detection result in the binary classification data set is a value for representing whether there is an affinity, and an affinity detection result in the affinity regression data set is a value for representing a size of the affinity. In a testing process, the affinity prediction model provided in some embodiments is separately compared with an affinity prediction model provided in the related art. The affinity prediction model provided in the related art includes a machine learning-based affinity prediction model. An affinity is predicted by using methods such as a CNN and integrated learning. The affinity prediction model provided in the related art further includes a non-pretrained BERT model.

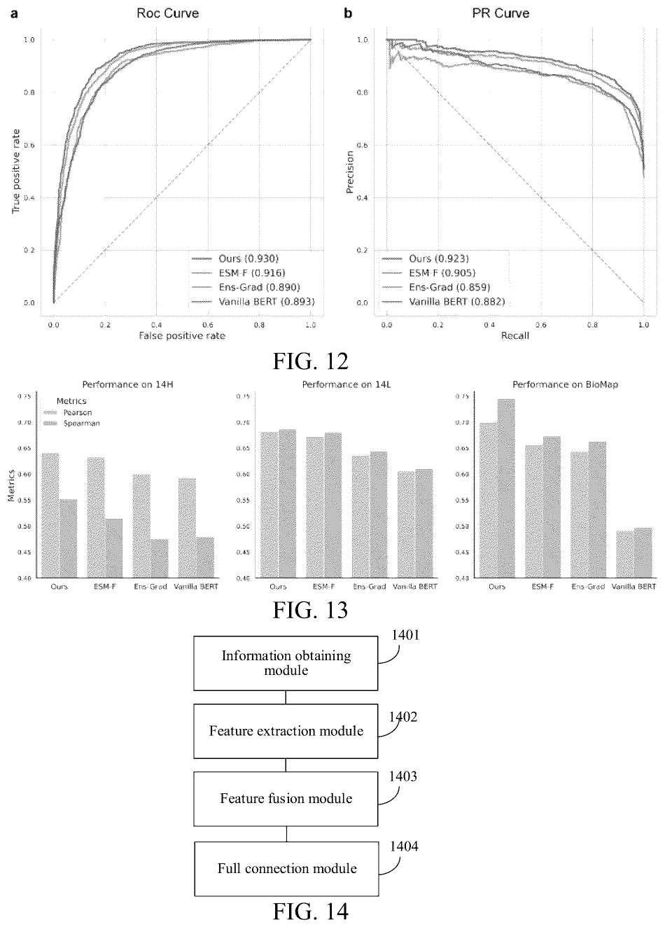

FIG. 12 is a schematic diagram of a performance detection result according to some embodiments. Performance detection is performed by using a binary classification data set. As shown in FIG. 12, a receiver operating characteristic curve (Roc Curve) of each model is shown on the left. The Roc Curve is a curve for evaluating performance of a binary classification model. The Roc Curve uses a true positive rate as a vertical axis and a false positive rate as a horizontal axis, where the true positive rate represents a rate at which a binary classification model correctly classifies positive examples, and the false positive rate represents a rate at which the binary classification model incorrectly classifies negative examples as positive examples. An “Ours” curve in FIG. 12 represents a Roc Curve of the affinity prediction model provided in some embodiments, which may reach 0.930 at most, and remaining curves are Roc Curves of the affinity prediction model provided in the related art. As can be seen from the figure, the Roc Curve of the affinity prediction model provided in some embodiments is significantly better than the Roc Curve of the affinity prediction model provided in the related art. As shown in FIG. 12, a precision-recall curve (PR Curve) of each model is shown on the right. The PR Curve uses coverage as a horizontal axis and precision as a vertical axis. An “Ours” curve in FIG. 12 represents a PR Curve of the affinity prediction model provided in some embodiments, which may reach 0.923 at most, and remaining curves are PR Curves of the affinity prediction model provided in the related art. As can be seen from the figure, the PR Curve of the affinity prediction model provided in some embodiments is significantly better than the PR Curve of the affinity prediction model provided in the related art.

FIG. 13 is a schematic diagram of another performance detection result according to some embodiments. Performance detection is performed by using an affinity regression data set. FIG. 13 separately shows performance detection results of a plurality of models on three data sets, and vertical coordinates are a Pearson correlation coefficient (a correlation coefficient for measuring a linear relationship) and a Spearman correlation coefficient (a correlation coefficient for measuring a dependency). An “Ours” curve in FIG. 13 represents the affinity prediction model provided in some embodiments. As can be seen from FIG. 13, on the three data sets, the Pearson correlation coefficient and the Spearman correlation coefficient of the affinity prediction model provided in some embodiments both exceed the Pearson correlation coefficient and the Spearman correlation coefficient of the affinity prediction model provided in the related art.

FIG. 14 is a schematic diagram of a structure of an apparatus for predicting an affinity of an antibody sequence for an antigen sequence according to some embodiments. Referring to FIG. 14, the apparatus includes:
an information obtaining module 1401, configured to obtain antigen sequence information and antibody sequence information, where the antibody sequence information includes light chain sequence information and heavy chain sequence information of an antibody sequence, the antigen sequence information represents an amino acid in an antigen sequence, the light chain sequence information represents an amino acid in a light chain of the antibody sequence, and the heavy chain sequence information represents an amino acid in a heavy chain of the antibody sequence;
a feature extraction module 1402, configured to extract an antigen sequence feature from the antigen sequence information, extract a light chain sequence feature from the light chain sequence information, and extract a heavy chain sequence feature from the heavy chain sequence information;
a feature fusion module 1403, configured to fuse the antigen sequence feature, the light chain sequence feature, and the heavy chain sequence feature, to obtain fused sequence features;
a full connection module 1404, configured to fully connect the fused sequence features, to obtain an affinity detection result, where the affinity detection result represents an affinity of the antibody sequence for the antigen sequence.

In the apparatus for predicting an affinity of an antibody sequence for an antigen sequence according to some embodiments, when an affinity of an antibody sequence for an antigen sequence is predicted, a feature of an amino acid in the antigen sequence, a feature of an amino acid in a light chain of the antibody sequence, and a feature of an amino acid in a heavy chain of the antibody sequence are comprehensively considered. In terms of the heavy chain and the light chain, potential associations of the feature of the amino acid in the antigen sequence with the feature of the amino acid in the light chain and the feature of the amino acid in the heavy chain are considered, so as to predict the affinity of the antibody sequence for the antigen sequence. Not only factors are considered comprehensively, but also two granularities of the heavy chain and the light chain are divided, which is beneficial to improving the accuracy of affinity prediction.

In some embodiments, the affinity detection result is obtained by using an affinity prediction model. The affinity prediction model includes an antigen encoding network, a light chain encoding network, and a heavy chain encoding network. The feature extraction module 1402 is configured to:
extract the antigen sequence feature from the antigen sequence information by using the antigen encoding network;
extract the light chain sequence feature from the light chain sequence information by using the light chain encoding network; and
extract the heavy chain sequence feature from the heavy chain sequence information by using the heavy chain encoding network.

In some embodiments, the feature extraction module 1402 is configured to:
determine a first semantic feature and a first spatial feature of the antigen sequence information by using the antigen encoding network, where the first semantic feature represents features of a plurality of amino acids in the antigen sequence information, and the first spatial feature represents features of positions of the plurality of amino acids in the antigen sequence information; and generate the antigen sequence feature according to the first semantic feature and the first spatial feature.

In some embodiments, the feature extraction module 1402 is configured to:
perform feature extraction on the first semantic feature and the first spatial feature, to obtain a first key feature, a first value feature, and a first query feature;
fuse the first key feature, the first value feature, and the first query feature, to obtain a candidate antigen sequence feature; and
convert the candidate antigen sequence feature repeatedly, to obtain the antigen sequence feature.

In some embodiments, the feature extraction module 1402 is configured to:
determine a second semantic feature and a second spatial feature of the light chain sequence information by using the light chain encoding network, where the second semantic feature represents features of a plurality of amino acids in the light chain sequence information, and the second spatial feature represents features of positions of the plurality of amino acids in the light chain sequence information; and generate the light chain sequence feature according to the second semantic feature and the second spatial feature.

In some embodiments, the feature extraction module 1402 is configured to:
perform feature extraction on the second semantic feature and the second spatial feature, to obtain a second key feature, a second value feature, and a second query feature;
fuse the second key feature, the second value feature, and the second query feature, to obtain a candidate light chain sequence feature; and
convert the candidate light chain sequence feature repeatedly, to obtain the light chain sequence feature.

In some embodiments, the feature extraction module 1402 is configured to:
determine a third semantic feature and a third spatial feature of the heavy chain sequence information by using the heavy chain encoding network, where the third semantic feature represents features of a plurality of amino acids in the heavy chain sequence information, and the third spatial feature represents features of positions of the plurality of amino acids in the heavy chain sequence information; and generate the heavy chain sequence feature according to the third semantic feature and the third spatial feature.

In some embodiments, the feature extraction module 1402 is configured to:
perform feature extraction on the third semantic feature and the third spatial feature, to obtain a third key feature, a third value feature, and a third query feature;
fuse the third key feature, the third value feature, and the third query feature, to obtain a candidate heavy chain sequence feature; and
convert the candidate heavy chain sequence feature repeatedly, to obtain the heavy chain sequence feature.

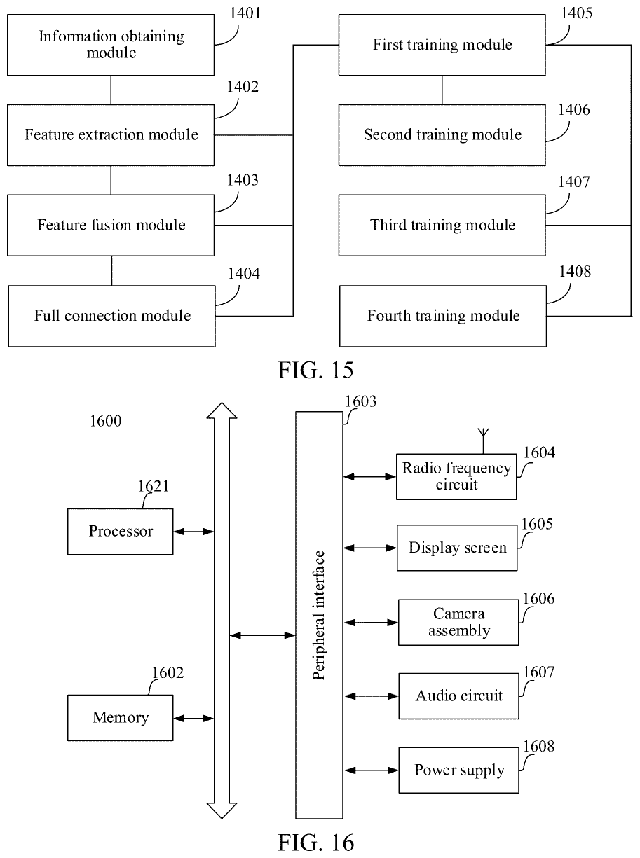

In some embodiments, referring to FIG. 15, the apparatus further includes a first training module 1405, configured to:
obtain sample antigen sequence information, sample antibody sequence information, and a real affinity detection result, where the sample antibody sequence information includes sample light chain sequence information and sample heavy chain sequence information, and the real affinity detection result represents a real affinity of the sample antibody sequence for the sample antigen sequence;
respectively perform feature extraction on the sample antigen sequence information, the sample light chain sequence information, and the sample heavy chain sequence information by using the affinity prediction model, to obtain a sample antigen sequence feature, a sample light chain sequence feature, and a sample heavy chain sequence feature; fuse the sample antigen sequence feature, the sample light chain sequence feature, and the sample heavy chain sequence feature, to obtain sample fused sequence features; fully connect the sample fused sequence features, to obtain a sample affinity detection result; and
train the affinity prediction model based on the sample affinity detection result and the real affinity detection result.

In some embodiments, referring to FIG. 15, the antigen encoding network is a trained network. The apparatus further includes a second training module 1406, configured to:
obtain first antigen sequence information, and mask amino acid information at a part of positions in the first antigen sequence information, to obtain second antigen sequence information;
predict, by using the antigen encoding network, the amino acid information at masked positions in the second antigen sequence information, to obtain a first predicted probability, where the first predicted probability represents a predicted probability that amino acids at the masked positions in the first antigen sequence information belong to each amino acid; and
train the antigen encoding network based on the first predicted probability and a first real probability, where the first real probability represents a real probability that the amino acids at the masked positions in the first antigen sequence information belong to each amino acid.

In some embodiments, referring to FIG. 15, the light chain encoding network is a trained network. The apparatus further includes a third training module 1407, configured to:
obtain first light chain sequence information, and mask amino acid information at a part of positions in the first light chain sequence information, to obtain second light chain sequence information;
predict, by using the light chain encoding network, the amino acid information at masked positions in the second light chain sequence information, to obtain a second predicted probability, where the second predicted probability represents a predicted probability that amino acids at the masked positions in the first light chain sequence information belong to each amino acid; and
train the light chain encoding network based on the second predicted probability and a second real probability, where the second real probability represents a real probability that the amino acids at the masked positions in the first light chain sequence information belong to each amino acid.

In some embodiments, referring to FIG. 15, the heavy chain encoding network is a trained network. The apparatus further includes a fourth training module 1408, configured to:
obtain first heavy chain sequence information, and mask amino acid information at a part of positions in the first heavy chain sequence information, to obtain second heavy chain sequence information;
predict, by using the heavy chain encoding network, the amino acid information at masked positions in the second heavy chain sequence information, to obtain a third predicted probability, where the third predicted probability represents a predicted probability that amino acids at the masked positions in the first heavy chain sequence information belong to each amino acid; and
train the heavy chain encoding network based on the third predicted probability and a third real probability, where the third real probability represents a real probability that the amino acids at the masked positions in the first heavy chain sequence information belong to each amino acid.

The apparatus for predicting an affinity of an antibody sequence for an antigen sequence according to the foregoing embodiment is illustrated only with an example of division of the foregoing function modules. In practical applications, the foregoing functions may be allocated to and completed by different function modules according to requirements. To be specific, the internal structure of a computer device is divided into different function modules to complete all or some of the functions described above. In addition, the apparatus for predicting an affinity of an antibody sequence for an antigen sequence according to the foregoing embodiment and some embodiments of the method for predicting an affinity of an antibody sequence for an antigen sequence belong to a same conception.

Some embodiments further provides a computer device. The computer device includes a processor and a memory. The memory has at least one computer program stored therein. The at least one computer program is loaded and executed by the processor to implement the method for predicting an affinity of an antibody sequence for an antigen sequence according to the foregoing embodiment.

In some embodiments, the computer device is provided as a terminal. FIG. 16 is a schematic diagram of a structure of a terminal 1600 according to some embodiments. The terminal 1600 includes: a processor 1601 and a memory 1602.

The processor 1601 may include one or more processing cores, for example, a 4-core processor or an 8-core processor. The processor 1601 may be implemented in at least one hardware form of a digital signal processor (DSP), a field programmable gate array (FPGA), and a programmable logic array (PLA). The processor 1601 may include a main processor and a coprocessor. The main processor is configured to process data in a wake-up state, also referred to as a central processing unit (CPU). The coprocessor is a low-power processor configured to process data in a standby state. In some embodiments, the processor 1601 may be integrated with a graphics processing unit (GPU). The GPU is configured to render and draw content that may be displayed on a display screen. In some embodiments, the processor 1601 may further include an artificial intelligence (AI) processor. The AI processor is configured to process computing operations related to machine learning.

The memory 1602 may include one or more computer-readable storage media. The computer-readable storage medium may be non-transient. The memory 1602 may further include a high-speed random access memory and a nonvolatile memory, for example, one or more disk storage devices or flash storage devices. In some embodiments, the non-transient computer-readable storage medium in the memory 1602 is configured to store at least one computer program. The at least one computer program is used by the processor 1601 to implement the method for predicting an affinity of an antibody sequence for an antigen sequence according to the method embodiment of this application.

In some embodiments, the terminal 1600 further includes: a peripheral interface 1603 and at least one peripheral. The processor 1601, the memory 1602, and the peripheral interface 1603 may be connected through a bus or a signal cable. Each peripheral may be connected to the peripheral interface 1603 through a bus, a signal cable, or a circuit board. In some embodiments, the peripheral include: at least one of a radio frequency (RF) circuit 1604, a display screen 1605, a camera assembly 1606, an audio circuit 1607, and a power supply 1608.

The peripheral interface 1603 may be configured to connect the at least one peripheral related to input/output (I/O) to the processor 1601 and the memory 1602. In some embodiments, the processor 1601, the memory 1602, and the peripheral interface 1603 are integrated on a same chip or circuit board. In some other embodiments, any or both of the processor 1601, the memory 1602, and the peripheral interface 1603 may be implemented on an independent chip or circuit board. This is not limited in some embodiments.

The RF circuit 1604 is configured to receive and transmit an RF signal, also referred to as an electromagnetic signal. The RF circuit 1604 communicates with a communication network and other communication devices through the electromagnetic signal. The RF circuit 1604 converts an electric signal into an electromagnetic signal for transmission, or converts a received electromagnetic signal into an electric signal. In some embodiments, the RF circuit 1604 includes: an antenna system, an RF transceiver, one or more amplifiers, a tuner, an oscillator, a DSP, a codec chipset, a user identity module card, and the like. The RF circuit 1604 may communicate with another device through at least one wireless communication protocol. The wireless communication protocol includes, but is not limited to, a metropolitan area network, generations of mobile communication networks (2G, 3G, 4G, and 5G), a wireless local area network and/or a wireless fidelity (WiFi) network. In some embodiments, the RF circuit 1604 may further include a circuit related to near field communication (NFC). This is not limited in this application.

The display screen 1605 is configured to display a user interface (UI). The UI may include a graph, text, an icon, a video, and any combination thereof. When the display screen 1605 is a touch display screen, the display screen 1605 further has a capability of acquiring a touch signal on or above a surface of the display screen 1605. The touch signal may be inputted to the processor 1601 as a control signal for processing. In this case, the display screen 1605 may be further configured to provide a virtual button and/or a virtual keyboard that are/is also referred to as a soft button and/or a soft keyboard. In some embodiments, one display screen 1605 may be disposed on a front panel of the terminal 1600. In some other embodiments, at least two display screens 1605 may be respectively disposed on different surfaces of the terminal 1600 or in a folded design. In some other embodiments, the display screen 1605 may be a flexible display screen disposed on a curved surface or a folded surface of the terminal 1600. Even, the display screen 1605 may be further set in a non-rectangular irregular pattern, namely, a special-shaped screen. The display screen 1605 may be prepared by using a material such as a liquid crystal display (LCD) or an organic light-emitting diode (OLED).

The camera assembly 1606 is configured to acquire images or videos. In some embodiments, the camera assembly 1606 includes a front camera and a rear camera. The front camera is disposed on the front panel of the terminal 1600. The rear camera is disposed on a rear surface of the terminal 1600. In some embodiments, there are at least two rear cameras, which are respectively any of a main camera, a depth-of-field camera, a wide-angle camera, and a telephoto camera, to achieve background blur through fusion of the main camera and the depth-of-field camera, panoramic photographing and virtual reality (VR) photographing through fusion of the main camera and the wide-angle camera, or other fusion photographing functions. In some embodiments, the camera assembly 1606 may further include a flash. The flash may be a monochrome temperature flash, or may be a double color temperature flash. The double color temperature flash refers to a combination of a warm light flash and a cold light flash, and may be used for light compensation under different color temperatures.

The audio circuit 1607 may include a microphone and a speaker. The microphone is configured to acquire sound waves of a user and an environment, and convert the sound waves into an electrical signal to input to the processor 1601 for processing, or input to the radio frequency circuit 1604 for implementing voice communication. For the purpose of stereo acquisition or noise reduction, a plurality of microphones may be respectively disposed at different portions of the terminal 1600. The microphone may further be an array microphone or an omni-directional acquisition type microphone. The speaker is configured to convert electric signals from the processor 1601 or the RF circuit 1604 into sound waves. The speaker may be a film speaker, or may be a piezoelectric ceramic speaker. When the speaker is the piezoelectric ceramic speaker, the speaker not only can convert an electric signal into acoustic waves audible to a human being, but also can convert an electric signal into acoustic waves inaudible to a human being, for ranging and other purposes. In some embodiments, the audio circuit 1607 may further include an earphone jack.

The power supply 1608 is configured to supply power to assemblies in the terminal 1600. The power supply 1608 may be an alternating current, a direct current, a primary battery, or a rechargeable battery. When the power supply 1608 includes a rechargeable battery, the rechargeable battery may support wired charging or wireless charging. The rechargeable battery may be further configured to support a fast charging technology.

A person skilled in the art may understand that the structure shown in FIG. 16 constitutes no limitation on the terminal 1600, and the terminal may include more or fewer assemblies than those shown in the figure, or some assemblies may be combined, or a different assembly deployment may be used.

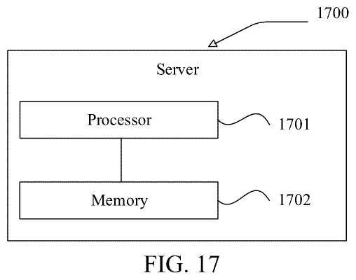

In some embodiments, the computer device is provided as a server. FIG. 17 is a schematic diagram of a structure of a server according to some embodiments. The server 1700 may vary greatly due to different configurations or performance, and may include one or more CPUs 1701 and one or more memories 1702. The memory 1702 has at least one computer program stored therein. The at least one computer program is loaded and executed by the processor 1701 to implement the methods provided in the foregoing method embodiments. It is clear that the server may further include components such as a wired or wireless network interface, a keyboard, and an I/O interface, to perform input and output. The server may further include another component configured to implement a device function. Details are not described herein.

Some embodiments further provides a computer-readable storage medium. The computer-readable storage medium has at least one computer program stored therein. The at least one computer program is loaded and executed by a processor to implement the method for predicting an affinity of an antibody sequence for an antigen sequence according to the foregoing embodiment.

Some embodiments further provides a computer program product, including a computer program. The computer program is loaded and executed by a processor to implement the method for predicting an affinity of an antibody sequence for an antigen sequence in the foregoing embodiment.

Technical features of the foregoing embodiments may be randomly combined. To make description concise, not all possible combinations of the technical features in the foregoing embodiments are described. However, the combinations of these technical features shall be considered as falling within the scope recorded by this specification provided that no conflict exists.

The foregoing embodiments only describe several implementations of this application, which are described and in detail, but cannot be construed as a limitation to the patent scope of this application. For a person of ordinary skill in the art, several transformations and improvements may be made without departing from the conception of this application. These transformations and improvements belong to the protection scope of this application. Therefore, the protection scope of the patent of this application shall be subject to the appended claims.

What is claimed is:

1. A method for predicting an affinity of an antibody sequence for an antigen sequence, performed by a computer device, the method comprising:
obtaining antigen sequence information representing an amino acid in an antigen sequence;
obtaining antibody sequence information comprising light chain sequence information and heavy chain sequence information of an antibody sequence,
wherein the light chain sequence information represents an amino acid in a light chain of the antibody sequence, and the heavy chain sequence information represents amino acid in a heavy chain of the antibody sequence;
extracting an antigen sequence feature from the antigen sequence information;
extracting a light chain sequence feature from the light chain sequence information;
extracting a heavy chain sequence feature from the heavy chain sequence information;
fusing the antigen sequence feature, the light chain sequence feature, and the heavy chain sequence feature, to obtain fused sequence features; and
processing the fused sequence features based on a fully connected neural network;
determining an affinity detection result representing an affinity of the antibody sequence for the antigen sequence.

2. The method according to claim 1, the method further comprising:
extracting the antigen sequence feature from the antigen sequence information based on an antigen encoding network;
extracting the light chain sequence feature from the light chain sequence information based on a light chain encoding network;
extracting the heavy chain sequence feature from the heavy chain sequence information based on a heavy chain encoding network;
determining the affinity detection result based on an affinity prediction model, the affinity prediction model comprising the antigen encoding network, the light chain encoding network, and the heavy chain encoding network.

3. The method according to claim 2, wherein the extracting the antigen sequence feature from the antigen sequence information based on the antigen encoding network comprises:
determining a first semantic feature and a first spatial feature of the antigen sequence information based on the antigen encoding network,
wherein the first semantic feature represents features of a plurality of amino acids in the antigen sequence information, and the first spatial feature represents features of positions of the plurality of amino acids; and
generating the antigen sequence feature based on the first semantic feature and the first spatial feature.

4. The method according to claim 3, wherein the generating the antigen sequence feature based on the first semantic feature and the first spatial feature comprises:
extracting, from the first semantic feature and the first spatial feature, a first key feature, a first value feature, and a first query feature;
fusing the first key feature, the first value feature, and the first query feature, to obtain a candidate antigen sequence feature; and
iteratively processing the candidate antigen sequence feature, to obtain the antigen sequence feature.

5. The method according to claim 2, wherein the extracting the light chain sequence feature from the light chain sequence information based on the light chain encoding network comprises:
determining a second semantic feature and a second spatial feature of the light chain sequence information based on the light chain encoding network,
wherein the second semantic feature represents features of a plurality of amino acids in the light chain sequence information, and the second spatial feature represents features of positions of the plurality of amino acids in the light chain sequence information; and
generating the light chain sequence feature based on the second semantic feature and the second spatial feature.

6. The method according to claim 5, wherein the generating the light chain sequence feature based on the second semantic feature and the second spatial feature comprises:
extracting, from the second semantic feature and the second spatial feature, a second key feature, a second value feature, and a second query feature;
fusing the second key feature, the second value feature, and the second query feature, to obtain a candidate light chain sequence feature; and
iteratively processing the candidate light chain sequence feature, to obtain the light chain sequence feature.

7. The method according to claim 2, wherein the extracting the heavy chain sequence feature from the heavy chain sequence information based on the heavy chain encoding network comprises:
determining a third semantic feature and a third spatial feature of the heavy chain sequence information based on the heavy chain encoding network,
wherein the third semantic feature represents features of a plurality of amino acids in the heavy chain sequence information, and the third spatial feature represents features of positions of the plurality of amino acids in the heavy chain sequence information; and
generating the heavy chain sequence feature based on the third semantic feature and the third spatial feature.

8. The method according to claim 7, wherein the generating the heavy chain sequence feature based on the third semantic feature and the third spatial feature comprises:
extracting from the third semantic feature and the third spatial feature, to obtain a third key feature, a third value feature, and a third query feature;
fusing the third key feature, the third value feature, and the third query feature, to obtain a candidate heavy chain sequence feature; and
iteratively processing the candidate heavy chain sequence feature, to obtain the heavy chain sequence feature.

9. The method according to claim 2, the method further comprising:
obtaining sample antibody sequence information that comprises sample light chain sequence information and sample heavy chain sequence information;
obtaining real affinity detection result that represents a real affinity of a sample antibody sequence for a sample antigen sequence;
extracting a sample antigen sequence feature, a sample light chain sequence feature, and a sample heavy chain sequence feature, from the sample antigen sequence information, the sample light chain sequence information, and the sample heavy chain sequence information based on the affinity prediction model;
fusing the sample antigen sequence feature, the sample light chain sequence feature, and the sample heavy chain sequence feature, to obtain sample fused sequence features;
processing the sample fused sequence features based on a fully connected neural network, to obtain a sample affinity detection result; and
training the affinity prediction model based on the sample affinity detection result and the real affinity detection result.

10. The method according to claim 2,
wherein the light chain encoding network is a trained network, and the method further comprising:
obtaining first light chain sequence information;
masking amino acid information at selected positions in the first light chain sequence information to obtain second light chain sequence information;
predicting, based on the light chain encoding network, the amino acid information at masked positions in the second light chain sequence information, to obtain a predicted probability of amino acids at the masked positions in the first light chain sequence information belonging to each amino acid; and
training the light chain encoding network based on the predicted probability and a corresponding real probability.

11. The method according to claim 2,
wherein the heavy chain encoding network is a trained network, the method further comprising:
obtaining first heavy chain sequence information;
masking amino acid information at selected positions in the first heavy chain sequence information, to obtain second heavy chain sequence information;
predicting, based on the heavy chain encoding network, the amino acid information at masked positions in the second heavy chain sequence information, to obtain a predicted probability of amino acids at the masked positions in the first heavy chain sequence information belonging to each amino acid; and
training the heavy chain encoding network based on the predicted probability and a corresponding real probability.

12. An apparatus for predicting an affinity of an antibody sequence for an antigen sequence, comprising:
at least one memory configured to store program code; and
at least one processor configured to read the program code and operate as instructed by the program code, the program code comprising:
obtaining code configured to cause at least one of the at least one processor to obtain antigen sequence information representing an amino acid in an antigen sequence;
obtaining code configured to cause at least one of the at least one processor to obtain antibody sequence information comprising light chain sequence information and heavy chain sequence information of an antibody sequence,
wherein the light chain sequence information represents an amino acid in a light chain of the antibody sequence, and the heavy chain sequence information represents an amino acid in a heavy chain of the antibody sequence;
extracting code configured to cause at least one of the at least one processor to extract an antigen sequence feature from the antigen sequence information;
extracting code configured to cause at least one of the at least one processor to extract a light chain sequence feature from the light chain sequence information;
extracting code configured to cause at least one of the at least one processor to extract a heavy chain sequence feature from the heavy chain sequence information;
fusing code configured to cause at least one of the at least one processor to fuse the antigen sequence feature, the light chain sequence feature, and the heavy chain sequence feature, to obtain fused sequence features; and
processing code configured to cause at least one of the at least one processor to process the fused sequence features based on a fully connected neural network;
determining code configured to cause at least one of the at least one processor to determine an affinity detection result representing an affinity of the antibody sequence for the antigen sequence.

13. The apparatus according to claim 12, wherein the program code is further configured to cause at least one of the at least one processor to:
extract the antigen sequence feature from the antigen sequence information based on an antigen encoding network;
extract the light chain sequence feature from the light chain sequence information based on a light chain encoding network;
extract the heavy chain sequence feature from the heavy chain sequence information based on a heavy chain encoding network;
determine the affinity detection result based on an affinity prediction model, the affinity prediction model comprising the antigen encoding network, the light chain encoding network, and the heavy chain encoding network.

14. The apparatus according to claim 13, wherein the extracting code is further configured to cause at least one of the at least one processor to:
determine a first semantic feature and a first spatial feature of the antigen sequence information based on the antigen encoding network,
wherein the first semantic feature represents features of a plurality of amino acids in the antigen sequence information, and the first spatial feature represents features of positions of the plurality of amino acids; and
generate the antigen sequence feature based on the first semantic feature and the first spatial feature.

15. The apparatus according to claim 14, wherein the extracting code is further configured to cause at least one of the at least one processor to:
extract, from the first semantic feature and the first spatial feature, a first key feature, a first value feature, and a first query feature;
fuse the first key feature, the first value feature, and the first query feature, to obtain a candidate antigen sequence feature; and
iteratively process the candidate antigen sequence feature, to obtain the antigen sequence feature.

16. The apparatus according to claim 13, wherein the extracting code is further configured to cause at least one of the at least one processor to:
determine a second semantic feature and a second spatial feature of the light chain sequence information based on the light chain encoding network,
wherein the second semantic feature represents features of a plurality of amino acids in the light chain sequence information, and the second spatial feature represents features of positions of the plurality of amino acids in the light chain sequence information; and
generate the light chain sequence feature based on the second semantic feature and the second spatial feature.

17. The apparatus according to claim 16, wherein the extracting code is further configured to cause at least one of the at least one processor to:
extract, from the second semantic feature and the second spatial feature, a second key feature, a second value feature, and a second query feature;
fuse the second key feature, the second value feature, and the second query feature, to obtain a candidate light chain sequence feature; and
iteratively process the candidate light chain sequence feature, to obtain the light chain sequence feature.

18. The apparatus according to claim 13, wherein the extracting code is further configured to cause at least one of the at least one processor to:
determine a third semantic feature and a third spatial feature of the heavy chain sequence information based on the heavy chain encoding network,
wherein the third semantic feature represents features of a plurality of amino acids in the heavy chain sequence information, and the third spatial feature represents features of positions of the plurality of amino acids in the heavy chain sequence information; and
generate the heavy chain sequence feature based on the third semantic feature and the third spatial feature.

19. The apparatus according to claim 18, wherein the extracting code is further configured to cause at least one of the at least one processor to:
extract from the third semantic feature and the third spatial feature, to obtain a third key feature, a third value feature, and a third query feature;
fuse the third key feature, the third value feature, and the third query feature, to obtain a candidate heavy chain sequence feature; and
iteratively process the candidate heavy chain sequence feature, to obtain the heavy chain sequence feature.

20. A non-transitory computer-readable storage medium, storing computer code which, when executed by at least one processor, causes the at least one processor to at least:
obtain antigen sequence information representing an amino acid in an antigen sequence;
obtain antibody sequence information comprising light chain sequence information and heavy chain sequence information of an antibody sequence,
wherein the light chain sequence information represents an amino acid in a light chain of the antibody sequence, and the heavy chain sequence information represents an amino acid in a heavy chain of the antibody sequence;
extract an antigen sequence feature from the antigen sequence information;
extract a light chain sequence feature from the light chain sequence information;
extract a heavy chain sequence feature from the heavy chain sequence information;
fuse the antigen sequence feature, the light chain sequence feature.
